(19) United States
(12) Patent Application Publication
(10) Pub. No.: US 2010/0324279 A1
(43) Pub. Date: Dec. 23, 2010

(54) CRYSTAL FORMS OF 2-[2-(4-CHLOROPHENYL)ETHOXY]ADENOSINE

(76) Inventors: Patricia M.J. Andres, West Lafayette, IN (US); Ron C. Kelly, Haywird, CA (US); Allan R. Moorman, Durham, NC (US); Matthew Strange, Loogotee, IN (US)

Correspondence Address: KING PHARMACEUTICALS, INC., 400 CROSSING BOULEVARD, BRIDGEWATER, NJ, 08807, US

(21) Appl. No.: 12/742,768

(22) PCT Filed: Dec. 18, 2008
(86) PCT No.: PCT/US08/87357

§ 371 Date: Aug. 11, 2010

Related U.S. Application Data
(60) Provisional application No. 61/015,229, filed on Dec. 20, 2007.

(51) Int. Cl. C07H 19/167

(52) U.S. Cl. 536/27.8

(57) ABSTRACT

The present invention provides novel crystal forms of 2-[2-(4-chlorophenyl)ethoxy]adenosine of the formula processes for the production of such crystal forms, and methods for the manufacture of pharmaceutical compositions for the treatment of diseases or conditions modulated by the adenosine A2 receptors, in particular the A2A receptor, in a mammal in need thereof, by employing such crystal forms. The crystal forms of the present invention are especially useful in the preparation of topical compositions for accelerating wound healing, e.g., for the treatment of diabetic foot ulcers.

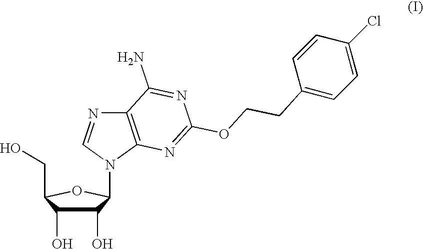

### FIELD OF THE INVENTION

The present invention provides crystal forms of 2-[2-(4-chlorophenyl)ethoxy]adenosine; processes for the production of such crystal forms; and methods for the manufacture of pharmaceutical compositions for treatment of diseases or conditions modulated by the adenosine A₂ receptors, in particular the A_{2A} receptor, by employing such crystal forms.

### BACKGROUND OF THE INVENTION

Adenosine is an endogenous nucleoside present in all cell types of the body. It is endogenously formed and released into the extracellular space under physiological and pathophysiological conditions characterized by an increased oxygen demand/supply ratio. This means that the formation of adenosine is accelerated in conditions with increased high energy phosphate degradation. The biological actions of adenosine are mediated through specific adenosine receptors located on the cell surface of various cell types.

The adenosine A₂ receptors, in particular the A_{2A} receptor, which are stimulatory to adenylyl cyclase activity, are widely distributed in the central nervous system (CNS), and peripheral tissues.

Adenosine and its analogues interact with neutrophils in inflammatory responses. While neutrophils are essential for limiting the spread of infection by a variety of microbes, stimulated neutrophils may damage injured tissues while en route to sites of infection or inflammation. Release of adenosine is one mechanism by which normal cells may protect themselves from activated neutrophils. Thus, one important action of adenosine and its analogues is to inhibit the generation of toxic oxygen metabolites, including O₂− and H₂O₂, by interacting with A₂ receptors on the neutrophils (Cronstein et al., J. Immunol. 135: 1366-1371, 1985; Roberts et al., Biochem. J. 227: 669-674, 1985). Furthermore, adenosine promotes neutrophil chemotaxis via the same receptors (Cronstein et al., supra; Rose et al., J. Exp. Med. 167: 1186-1194, 1988). Likewise, adenosine receptor ligation regulates inflammatory responses of neutrophils triggered by immune complexes acting through the Fcγ receptor (Salmon, J. Immunol. 145: 2235-2240, 1990).

In vitro studies have demonstrated that adenosine A₂ receptor agonists promote fibroblast and endothelial cell migration into an artificial wound (Montesinos et al., J. Exp. Med. 186(9): 1615-1620, 1997) and, therefore, development of adenosine A₂ receptor agonists for wound healing has become of particular interest.

Wound healing is a complex process characterized by three overlapping phases: inflammation, tissue formation, and tissue remodeling. During tissue formation, growth factors synthesized by local and migratory cells stimulate fibroblasts to migrate into the wound where they proliferate and construct an extracellular matrix. Chronic wound healing is characterized by additional complexities and conventional types of therapy are oftentimes inadequate for healing chronic wounds. Indeed chronic wounds resist healing and closure. It is not uncommon that wounds such as diabetic ulcers will become chronic open wounds (Wieman et al., Diabetes Care, 21(5), 822-827, 1998).

Recently, the activation of adenosine A_{2A} receptors by the adenosine A_{2A} receptor agonist, 2-[2-(4-chlorophenyl)ethoxy]adenosine has been found to promote wound healing of diabetic ulcers in healthy BALB/C mice (Victor-Vega et al., Inflammation, 26(1) 19-24, 2002).

U.S. Pat. No. 6,951,932 to Moorman discloses the synthesis of 2-aralkoxy- and 2-alkoxyadenosine derivatives, specifically the preparation of 2-[2-(4-chlorophenyl)ethoxy]adenosine. U.S. Patent Application Publication No. US 20060135466 to Moorman et al discloses an alternative synthetic route for the preparation of 2-aralkoxy- and 2-alkoxyadenosine derivatives, e.g., 2-[2-(4-chlorophenyl)ethoxy]adenosine.

### SUMMARY OF THE INVENTION

The present invention provides novel crystal forms of 2-[2-(4-chlorophenyl)ethoxy]adenosine of the formula

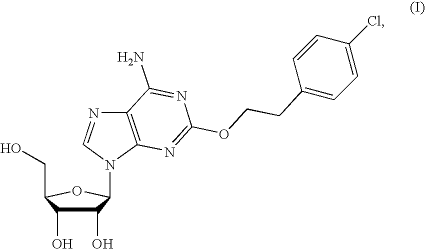

also known as MRE-0094; processes for the production of such crystal forms, and methods for the manufacture of pharmaceutical compositions for the treatment of diseases or conditions modulated by the adenosine A₂ receptors, in particular the A_{2A} receptor, in a mammal in need thereof, by employing such crystal forms. The crystal forms of the present invention are especially useful in the preparation of topical compositions for accelerating wound healing, e.g., for the treatment of diabetic foot ulcers.

Pharmaceuticals that exhibit polymorphism offer unique challenges in product development. Thus, it is essential to understand the polymorphic behavior of crystalline solids and their relative thermodynamic stability to avoid complications during processing and development. Conversion of one crystal form into unknown amounts of different crystalline or amorphous forms during processing or storage is undesirable, and in many cases would be regarded as analogous to the appearance of unquantified amounts of impurities in the product. Therefore, it is generally desirable to manufacture the drug substance in the most stable solid state form, thereby minimizing the possibility of less stable forms being generated during storage. However, the less stable solid state forms (polymorphs) may offer advantages over the most stable form, such as enhanced solubility, reduced hygroscopicity, and improved bulk properties e.g., improved flow properties and bulk density, any of which may make them more desirable than the most stable solid state form. These differences in physicochemical properties among the polymorphs of a drug substance are well known to those skilled in the art, and have been discussed widely in the literature (See for example “Polymorphism in Pharmaceutical Solids”, edited by Harry G. Brittain. Vol. 95, Drugs and the Pharmaceutical Sciences, Marcel Dekker, Inc 270 Madison Avenue, New York, N.Y. 10016. Copyright 1999).

Accordingly, there is a need to identify stable, e.g., crystalline forms of MRE-0094, which have good bulk properties and are easy to manage in the drying or grinding processes following the final stage of the chemical synthesis of the drug substance. The crystal forms of the present invention, in particular, the crystal form designated herein as the G-type crystal form, exhibit the desired improved properties as described herein.

Other objects, features, advantages and aspects of the present invention will become apparent to those skilled in the art from the following description, appended claims and accompanying drawings. It should be understood, however, that the following description, appended claims, drawings and specific examples, while indicating preferred embodiments of the present invention, are given by way of illustration only. Various changes and modifications within the spirit and scope of the disclosed invention will become readily apparent to those skilled in the art from reading the following.

### BRIEF DESCRIPTION OF THE DRAWINGS

FIG. 1 shows differential scanning calorimetry data of A-type crystal form of 2-[2-(4-chlorophenyl)ethoxy]adenosine;

FIG. 2 shows a powder X-ray diffraction diagram of A-type crystal form of 2-[2-(4-chlorophenyl)ethoxy]adenosine;

FIG. 3 shows an infrared absorption spectrum of A-type crystal form of 2-[2-(4-chlorophenyl)ethoxy]adenosine;

FIG. 4 shows a Raman spectrum of A-type crystal form of 2-[2-(4-chlorophenyl)ethoxy]adenosine;

FIG. 5 shows differential scanning calorimetry data of B-type crystal form of 2-[2-(4-chlorophenyl)ethoxy]adenosine;

FIG. 6 shows a powder X-ray diffraction diagram of B-type crystal form of 2-[2-(4-chlorophenyl)ethoxy]adenosine;

FIG. 7 shows an infrared absorption spectrum of B-type crystal form of 2-[2-(4-chlorophenyl)ethoxy]adenosine;

FIG. 8 shows a Raman spectrum of B-type crystal form of 2-[2-(4-chlorophenyl)ethoxy]adenosine;

FIG. 9 shows differential scanning calorimetry data of C-type crystal form of 2-[2-(4-chlorophenyl)ethoxy]adenosine;

FIG. 10 shows a powder X-ray diffraction diagram of C-type crystal form of 2-[2-(4-chlorophenyl)ethoxy]adenosine;

FIG. 11 shows differential scanning calorimetry data of D-type crystal form of 2-[2-(4-chlorophenyl)ethoxy]adenosine;

FIG. 12 shows a powder X-ray diffraction diagram of D-type crystal form of 2-[2-(4-chlorophenyl)ethoxy]adenosine;

FIG. 13 shows differential scanning calorimetry data of E-type crystal form of 2-[2-(4-chlorophenyl)ethoxy]adenosine;

FIG. 14 shows a powder X-ray diffraction diagram of E-type crystal form of 2-[2-(4-chlorophenyl)ethoxy]adenosine;

FIG. 15 shows differential scanning calorimetry data of F-type crystal form of 2-[2-(4-chlorophenyl)ethoxy]adenosine;

FIG. 16 shows a powder X-ray diffraction diagram of F-type crystal form of 2-[2-(4-chlorophenyl)ethoxy]adenosine;

FIG. 17 shows differential scanning calorimetry data of G-type crystal form of 2-[2-(4-chlorophenyl)ethoxy]adenosine;

FIG. 18 shows a powder X-ray diffraction diagram of G-type crystal form of 2-[2-(4-chlorophenyl)ethoxy]adenosine;

FIG. 19 shows an infrared absorption spectrum of G-type crystal form of 2-[2-(4-chlorophenyl)ethoxy]adenosine;

FIG. 20 shows a Raman spectrum of G-type crystal form of 2-[2-(4-chlorophenyl)ethoxy]adenosine;

FIG. 21 shows differential scanning calorimetry data of H-type crystal form of 2-[2-(4-chlorophenyl)ethoxy]adenosine;

FIG. 22 shows a powder X-ray diffraction diagram of H-type crystal form of 2-[2-(4-chlorophenyl)ethoxy]adenosine;

FIG. 23 shows differential scanning calorimetry data of I-type crystal form of 2-[2-(4-chlorophenyl)ethoxy]adenosine;

FIG. 24 shows a powder X-ray diffraction diagram of I-type crystal form of 2-[2-(4-chlorophenyl)ethoxy]adenosine;

FIG. 25 shows differential scanning calorimetry data of J-type crystal form of 2-[2-(4-chlorophenyl)ethoxy]adenosine;

FIG. 26 shows a powder X-ray diffraction diagram of J-type crystal form of 2-[2-(4-chlorophenyl)ethoxy]adenosine;

FIG. 27 shows differential scanning calorimetry data of K-type crystal form of 2-[2-(4-chlorophenyl)ethoxy]adenosine, and

FIG. 28 shows a powder X-ray diffraction diagram of K-type crystal form of 2-[2-(4-chlorophenyl)ethoxy]adenosine.

FIG. 29 shows a powder X-ray diffraction diagram of amorphous 2-[2-(4-chlorophenyl)ethoxy]adenosine.

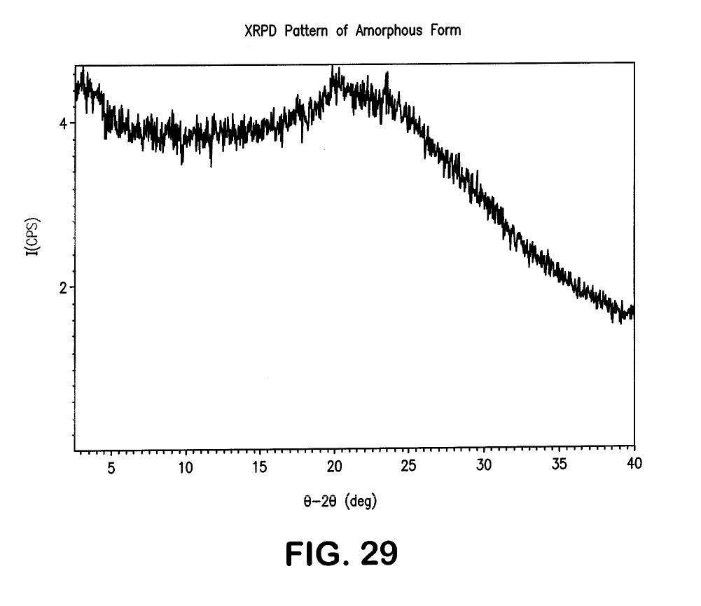

### DETAILED DESCRIPTION OF THE INVENTION

As described herein above, the present invention provides novel crystal forms of 2-[2-(4-chlorophenyl)ethoxy]adenosine of the formula

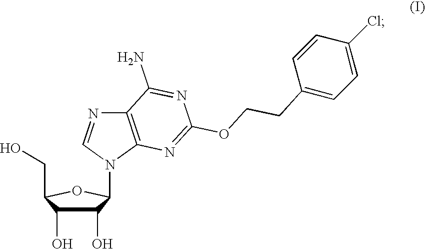

also known as MRE-0094; processes for the production of such crystal forms, and methods for the manufacture of pharmaceutical compositions for the treatment of diseases or conditions modulated by the adenosine A₂ receptors, in particular the A_{2A} receptor, in a mammal in need thereof, by employing such crystal forms. The crystal forms of the present invention are especially useful in the preparation of topical compositions for accelerating wound healing, e.g., for the treatment of diabetic foot ulcers.

As employed throughout the description and appended claims, the term “crystals” or “crystal forms” of the instant invention refers to, as appropriate, crystal forms of 2-[2-(4-chlorophenyl)ethoxy]adenosine, i.e., MRE-0094, designated as A-type, B-type, C-type, D-type, E-type, F-type, G-type, I-type, J-type and K-type crystal forms as defined herein below, and are substantially free of all other alternative crystalline and amorphous forms.

The term “substantially free” when referring to a designated crystal form of MRE-0094 means that the designated crystal form contains less than 20% (by weight) of any alternate polymorphic form(s) of MRE-0094, preferably less than 10% (by weight) of any alternate polymorphic form(s) of MRE-0094, more preferably less than 5% (by weight) of any alternate polymorphic form(s) of MRE-0094, and most preferably less than 1% (by weight) of any alternate polymorphic forms of MRE-0094

The crystal forms of the present invention may be characterized by measuring at least one of the following physico-chemical properties: 1) a melting point and/or thermal differential scanning calorimetry (DSC) data; 2) a X-ray powder diffraction pattern; 3) an infrared absorption spectrum; and 4) a Raman spectrum.

The melting points (m.p.) may be measured by differential scanning calorimetry (DSC) method using, e.g., a TA Instruments differential scanning calorimeter 2920. The sample is placed into an aluminium DSC pan, and the weight is recorded. The pan is covered with a lid and then crimped. The sample cell is equilibrated at 25° C. and heated under a nitrogen purge at a rate of 10 or 20° C./min, up to a final temperature of 350° C. A separate sample is used for a DSC cycling study. The sample is heated to 178° C., cooled to 25° C., and then heated to a final temperature of 350° C. at a rate of 10° C./min Indium metal is used as the calibration standard. Reported temperatures are at the transition onsets and maxima.

X-ray powder diffraction (XRPD) analyses may be performed using, e.g., a Shimadzu XRD-6000 X-ray powder diffractometer and an Inel XRG-3000 equipped with a CPS (Curved Position Sensitive) detector with a 28 range of 120°.

For the Shimadzu XRD-6000 X-ray powder diffractometer, Cu Kα radiation is used. The instrument is equipped with a fine focus X-ray tube. The tube voltage and amperage are set to 40 kV and 40 mA, respectively. The divergence and scattering slits are set at 1° and the receiving slit is set at 0.15 mm. Diffracted radiation is detected by a NaI scintillation detector. A theta-two theta continuous scan at 3°/min (0.4 sec/0.02° step) from 2.5 to 40° 2θ is used. A silicon standard is analyzed to check the instrument alignment Data are collected and analyzed using XRD-6000 v. 4.1. Samples are prepared for analysis by placing them in an aluminum holder with a flat silicon insert.

For the Inel XRG-3000, real time data are collected using Cu—Kα radiation starting at approximately 2.5° 2θ, at a resolution of 0.03° 2θ. The tube voltage and amperage are set to 40 kV and 30 mA, respectively. The pattern is displayed from 2.5-40° 2θ. Samples are prepared for analysis by packing them into thin-walled glass capillaries. The capillaries are sometimes sealed by heat, or with Critoseal. Each capillary is mounted onto a goniometer head that is motorized to permit spinning of the capillary during data acquisition. The samples are generally analyzed for 5 to 10 min Analysis time is 20-30 min for pattern indexing, which requires higher resolution Instrument calibration is performed using a silicon reference standard.

Infrared absorption spectra may be acquired, e.g., on a Magna-IR 960® Fourier transform infrared (FT-IR) spectrophotometer (Thermo Nicolet) equipped with an Ever-Glo mid/far IR source, an extended range potassium bromide (KBr) beamsplitter, and a deuterated triglycine sulfate (DIGS) detector. A diffuse reflectance accessory (the Collector™, Thermo Spectra-Tech) is used for sampling. Each spectrum represents 256 co-added scans collected at a spectral resolution of 4 cm⁻¹. Sample preparation consists of physically mixing the sample with KBr and placing the sample into a 13-mm macro cup and leveling the material. A background data set is acquired on a sample of KBr. A Log 1/R (R=reflectance) spectrum is acquired by taking a ratio of these two data sets against each other and converting it to Kubelka-Munk units. Wavelength calibration is performed using polystyrene.

FT-Raman spectra may be acquired, e.g., on a Raman accessory module interfaced to Magna-IR 960® FT-IR spectrometer (Thermo Nicolet). This spectrometer uses an excitation wavelength of 1064 nm. Approximately 0.972 to 0.988 W of Nd:YVO₄ laser power is used to irradiate the sample. The Raman spectra are measured with an indium gallium arsenide (InGaAs) detector. The samples are prepared for analysis by placing the material in a glass tube and positioning the tube in a gold-coated holder in the accessory. A total of 256 sample scans are collected from 3600 to 400 cm⁻¹ at a spectral resolution of 4 cm⁻¹ using Happ-Genzel apodization. Wavelength calibration is performed using sulfur and cyclohexane.

One of ordinary skill in the art will appreciate that the physico-chemical properties discussed herein above may be obtained with a measurement error that is dependent upon the measurement conditions employed. In particular, it is generally known that intensities in an X-ray diffraction pattern may fluctuate depending upon measurement conditions employed. It should be further understood that relative intensities may also vary depending upon experimental conditions and, accordingly, the exact order of intensity should not be taken into account. Additionally, a measurement error of diffraction angle for a conventional X-ray diffraction pattern is typically about 5% or less, e.g., ±0.2° 2θ, and such degree of measurement error should be taken into account as pertaining to the aforementioned diffraction angles. Consequently, it is to be understood that the crystal forms of the instant invention are not limited to the crystal forms that provide X-ray diffraction patterns completely identical to the X-ray diffraction patterns depicted in the accompanying Figures disclosed herein. Any crystal forms that provide X-ray diffraction patterns substantially identical to those disclosed in the accompanying Figures fall within the scope of the present invention. The ability to ascertain substantial identities of X-ray diffraction patterns is within the purview of one of ordinary skill in the art. A discussion of the theory of powder X-ray diffraction patterns can be found, e.g., in “X-Ray Diffraction Procedures” by Klug and Alexander, J. Wiley, New York (1974).

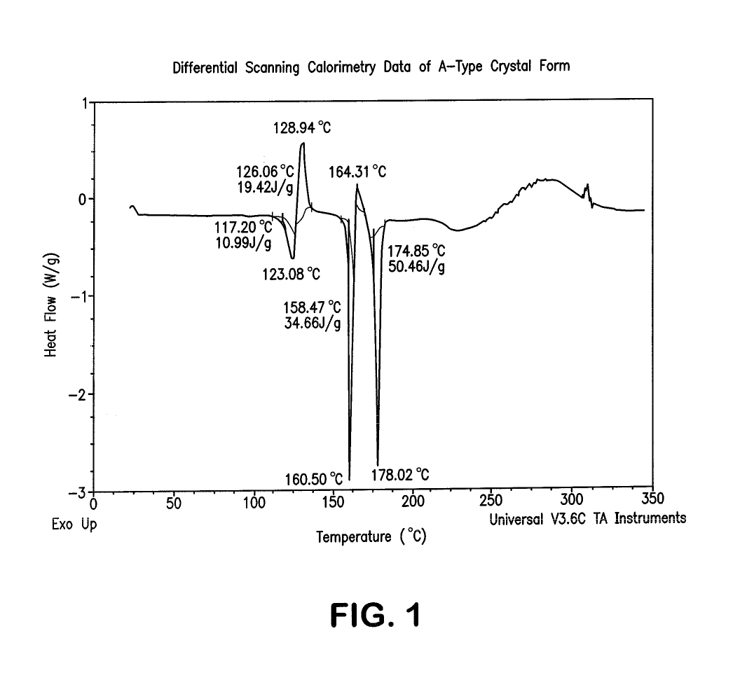

In one aspect, the present invention provides a crystal form of anhydrous MRE-0094, designated herein as A-type crystal form, that is characterized by thermal DSC data, as measured by the DSC method described herein above, substantially identical to those depicted in FIG. 1. A-Type crystals may be obtained, e.g., by crystallization from ethanol (EtOH).

Thermal DSC data of A-type crystals of MRE-0094 show several thermal events. An endothermic event is observed at an onset temperature of approximately 117° C. and a maximum temperature at about 123° C. when heated at 10° C./min, which is immediately followed by an exothermic event with an onset temperature of 126° C. A second endothermic event is observed at an onset temperature of 158° C. followed by a second exothermic event at 164° C. ending with a third endothermic event with an onset temperature of approximately 175° C. Thermogravimetric (TG) analysis shows negligible weight loss of approximately 0.8% between 25° C. and 220° C.

Thermogravimetric analyses (TG) may be performed using, e.g., a TA Instrument's 2950 thermogravimetric analyzer. Each sample is placed in an aluminium sample pan and inserted into the TG furnace. Samples are heated under nitrogen at a rate of 10° C./min. Nickel and Alumel™ are used as the calibration standards.

A DSC cycling study may be conducted in an attempt to identify the solid material that melts at approximately 178° C. The sample is heated to 178° C., cooled to 25° C., and then heated to a final temperature of 350° C. The first heating shows the thermal events that are observed in the initial DSC analysis. The second heating has only one endothermic event at a maximum temperature of 178° C. The single endothermic event observed during the second heating of the DSC cycling study coincides with the thermal behavior of G-type crystal form of MRE-0094

For heat stress studies, samples of MRE-0094 A-type crystals are heated isothermally at different temperatures to investigate the effect of temperatures on solid state conversions. The samples are heated for up to 15 min. The solid material is allowed to cool before analyzing by XRPD.

Heat stress studies of the A-type crystal form resulted in a solid form conversion at approximately 164° C. (2 min). Variable temperature XRPD (VT-XRPD) may be performed on A-type crystals of MRE-0094 to better understand its thermal behavior. The A-type crystal form of MRE-0094 remains as such up to 115° C. At 120° C., the A-type crystals convert to B-type crystal form. The sample remains as B-type crystals up to 165° C. At 165° C. the sample appears to be amorphous/low crystalline The recrystallization observed at approximately 164° C. from the DSC analysis is not captured in the VT-XRPD analysis. It is likely that the melting of B-type crystals, subsequent recrystallization, and final melting of the solid occurs too fast to be captured by VT-XRPD. Based on the VT-XRPD data, the thermal events observed by DSC at approximately 117° C. and 126° C. are due to the solid conversion followed by the subsequent recrystallization to form B-type crystals.

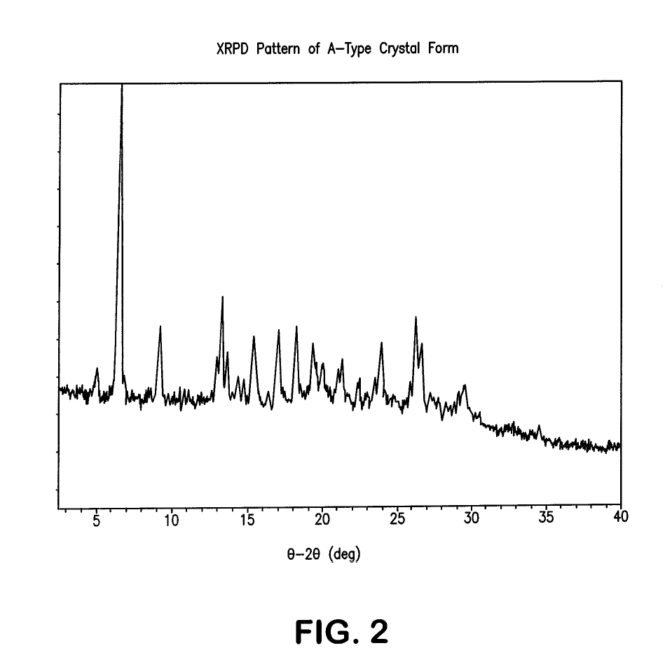

An example of an X-ray diffraction pattern exhibited by A-type crystal form is substantially identical to that depicted in FIG. 2, having characteristic peaks, expressed in degrees 2-theta (2θ), of about 6.4±0.2, 9.1±0.2, 13.2±0.2, 15.3±0.2, 17.0±0.2, 18.2±0.2 and 26.2±0.2. The present invention also provides A-type crystal form that exhibits an X-ray diffraction pattern substantially the same as that depicted in FIG. 2, having characteristic diffraction peaks expressed in degrees 2-theta, and relative intensities at approximately the values shown in Table 1 herein below

TABLE 1
A-Type Crystal Form of MRE-0094

| Angle (deg 2θ) | Relative Intensity (%) |
|---|---|
| 4.9 ± 0.2 | 37.5 |
| 6.4 ± 0.2 | 100.0 |
| 9.1 ± 0.2 | 50.1 |
| 12.9 ± 0.2 | 40.9 |
| 13.2 ± 0.2 | 58.2 |
| 13.6 ± 0.2 | 42.1 |
| 14.3 ± 0.2 | 35.0 |
| 14.7 ± 0.2 | 34.2 |
| 15.3 ± 0.2 | 46.5 |
| 17.0 ± 0.2 | 48.6 |
| 18.2 ± 0.2 | 49.9 |
| 19.3 ± 0.2 | 44.5 |
| 20.1 ± 0.2 | 38.7 |
| 21.1 ± 0.2 | 36.8 |
| 21.3 ± 0.2 | 39.8 |
| 22.5 ± 0.2 | 34.1 |
| 23.9 ± 0.2 | 44.7 |
| 24.8 ± 0.2 | 29.6 |
| 26.2 ± 0.2 | 51.8 |
| 26.5 ± 0.2 | 44.3 |
| 27.2 ± 0.2 | 30.0 |
| 27.5 ± 0.2 | 28.5 |
| 29.5 ± 0.2 | 32.3 |

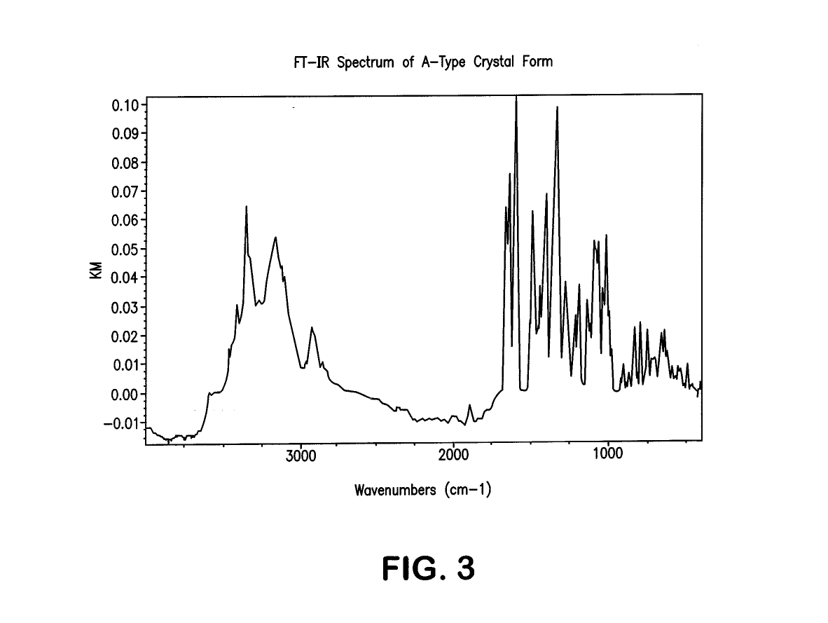

An example of an infrared absorption spectrum of A-type crystal form obtained by the KBr method is shown in FIG. 3, and is characterized by absorption bands at about 3360-3000, 1674±2, 1645±2 and 1600±2 cm⁻¹.

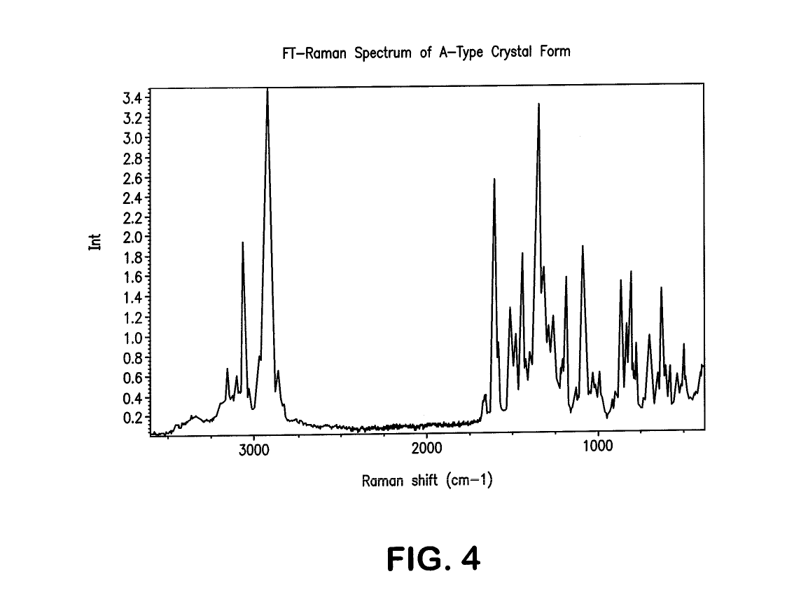

An example of a FT-Raman spectrum of A-type crystal form obtained by the method described herein above is shown in FIG. 4, and is characterized by Raman shifts at about 3160-3100, 2930±2, 1655±2, 1510±2 and 1440±2 cm⁻¹.

The A-type crystal form of MRE-0094 appears to be anhydrous, and moisture sorption analysis of the A-type crystals shows a total weight gain of approximately 4.3% (one mol of water) at 95% relative humidity (RH). The solid material gains approximately 0.3% of weight between 5 and 55% RH and an additional 4.0% of weight between 55% and 95% RH. The weight gain is lost upon drying, although some hysteresis is observed upon desorption Based on the moisture sorption data the A-type crystal form of MRE-0094 is hygroscopic and, therefore, may form crystalline hydrates. XRPD analysis of the solid material after moisture sorption identifies the material as A-type crystal form.

Moisture sorption/desorption analyses may be performed, e.g., on a VTI SGA-100 Vapor Sorption Analyzer Sorption and desorption data are collected, e.g., over a range of 5 to 95% relative humidity (RH) at 10% RH intervals under a nitrogen purge. Samples need not to be dried prior to analysis. Equilibrium criteria used for analysis are, e.g., less than 0.01% weight change in 5 min, with a maximum equilibration time of 3 h if the weight criterion is not met NaCl and PVP may used as calibration standards.

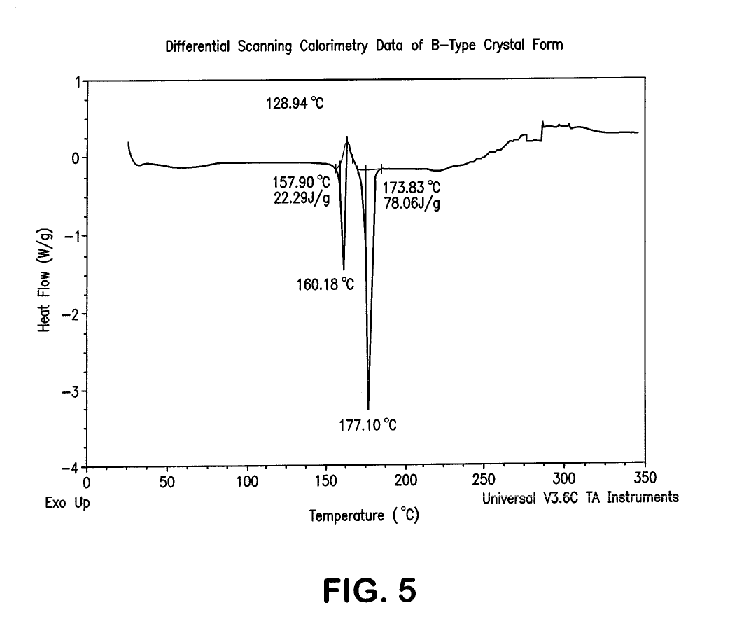

In another aspect, the present invention provides a crystal form of anhydrous MRE-0094, designated herein as B-type crystal form, that is characterized by thermal DSC data, as measured by the DSC method described herein above, substantially identical to those depicted in FIG. 5 B-Type crystals may be obtained by a variety of techniques, e.g., from a slurry in dichloromethane (DCM) or ethyl acetate (EtOAc).

Thermal DSC data for B-type crystal form of MRE-0094 shows an endothermic event with an onset temperature of 158° C. and a maximum temperature at about 160° C. when heated at 10° C./min, followed by an exothermic event at 163° C. A final endothermic event is observed at an onset temperature of 174° C. TG analysis shows approximately a 1% weight loss between 27° C. and 200° C. Significant weight loss is observed after 200° C., which may be due to decomposition.

The B-type crystal form of MRE-0094 appears to be anhydrous, and moisture sorption analysis of the B-type crystal form shows a total weight gain of approximately 3.9% (approximately one mol of water) between 5% and 95% relative humidity (RH). Approximately 3.7% of the weight is lost upon desorption. The solid material remaining after moisture sorption analysis is identified as B-type crystal form by XRPD. Based on the moisture sorption data, B-type crystal form appears to be a hygroscopic solid form when exposed to high relative humidity.

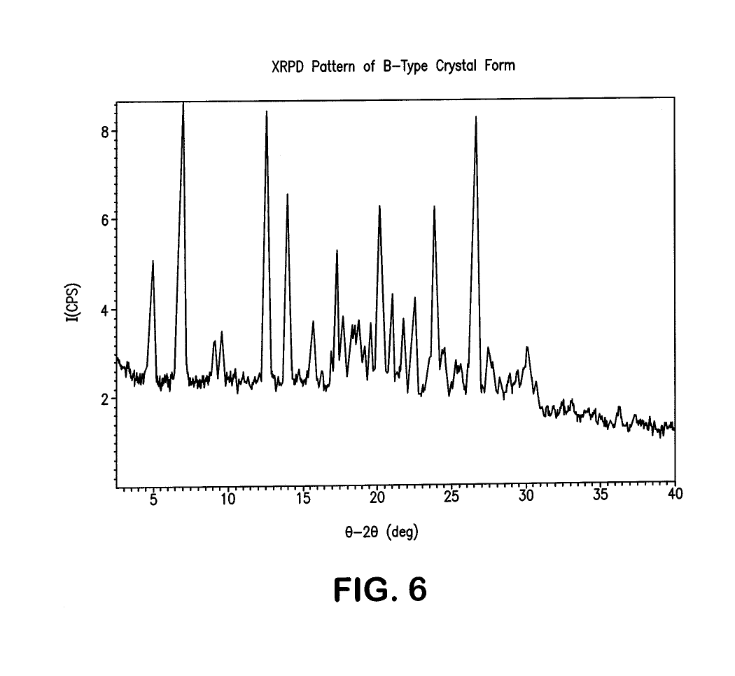

An example of an X-ray diffraction pattern exhibited by B-type crystal form is substantially identical to that depicted in FIG. 6, having characteristic peaks, expressed in degrees 2-theta (2θ), of about 4.9±0.2, 6.9±0.2, 12.5±0.2, 13.9±0.2, 17.2±0.2, 20.1±0.2, 23.8±0.2 and 26.5±0.2. The present invention also provides B-type crystal form that exhibits an X-ray diffraction pattern substantially the same as that depicted in FIG. 6, having characteristic diffraction peaks expressed in degrees 2-theta, and relative intensities at approximately the values shown in Table 2 herein below:

TABLE 2
B-Type Crystal Form of MRE-0094

| Angle (deg 2θ) | Relative Intensity (%) |
|---|---|
| 4.9 ± 0.2 | 59.1 |
| 6.9 ± 0.2 | 100.0 |
| 9.1 ± 0.2 | 38.0 |
| 9.5 ± 0.2 | 40.6 |
| 12.5 ± 0.2 | 98.0 |
| 13.9 ± 0.2 | 76.2 |
| 15.7 ± 0.2 | 43.5 |
| 17.2 ± 0.2 | 61.8 |
| 17.7 ± 0.2 | 44.5 |
| 19.5 ± 0.2 | 42.7 |
| 20.1 ± 0.2 | 73.1 |
| 21.0 ± 0.2 | 49.9 |
| 21.7 ± 0.2 | 43.4 |
| 22.5 ± 0.2 | 49.0 |
| 23.8 ± 0.2 | 73.5 |
| 26.5 ± 0.2 | 97.7 |
| 27.4 ± 0.2 | 35.6 |
| 30.1 ± 0.2 | 36.1 |

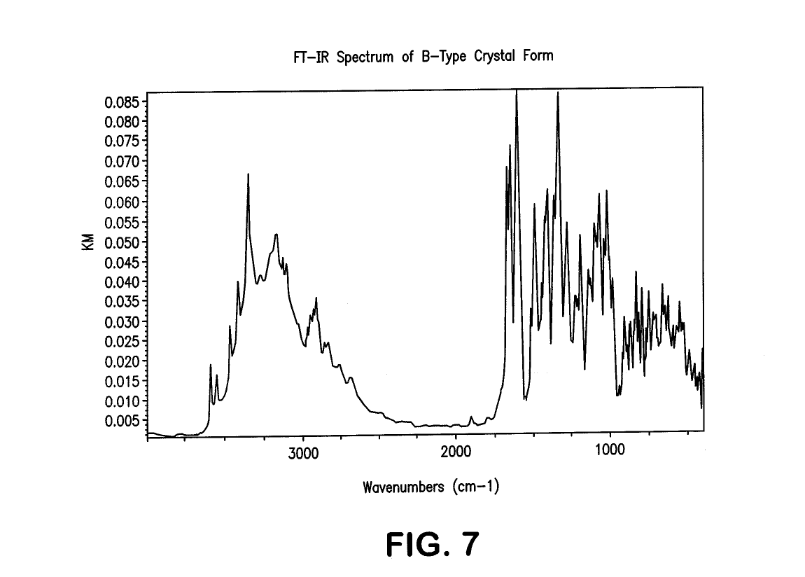

An example of an infrared absorption spectrum of B-type crystal form obtained by the KBr method is shown in FIG. 7, and is characterized by absorption bands at about 2985-2890, 2870-2825 and 2800-2755 cm⁻¹.

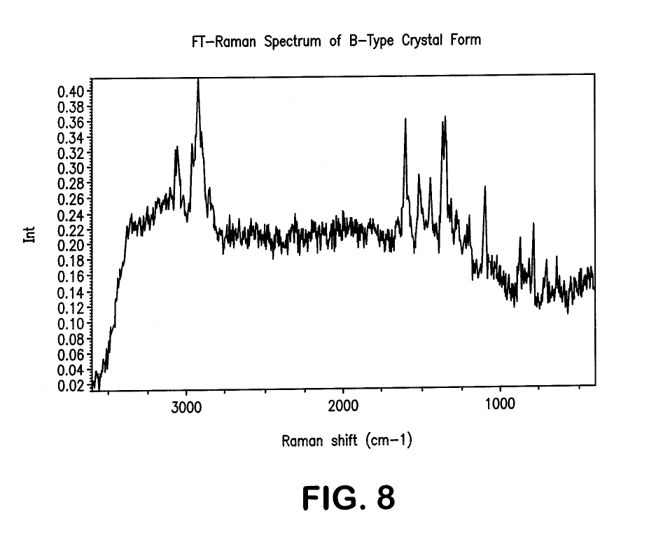

An example of a FT-Raman spectrum of B-type crystal form obtained by the method described herein above is shown in FIG. 8, and is characterized by Raman shifts at about 3080-3050 (doublet), 2970±2, 1360±2, 1360-1345 (doublet) and 1310±2 cm⁻¹.

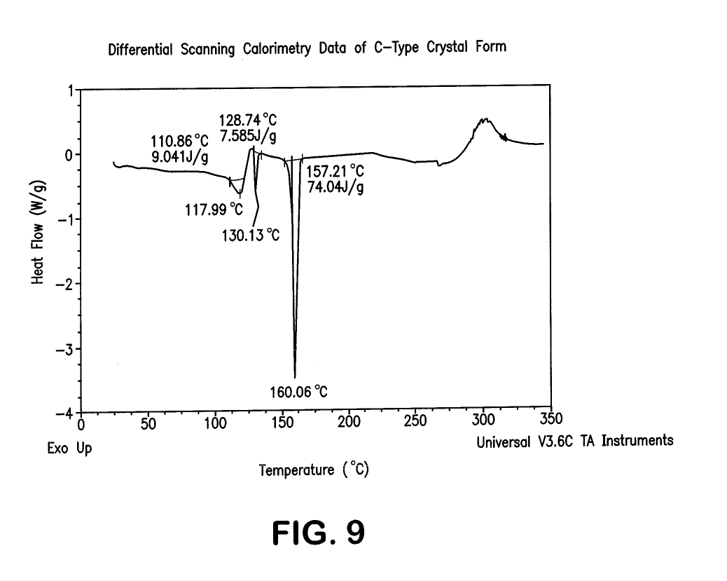

In yet another aspect, the present invention provides a crystal form of MRE-0094, designated herein as C-type crystal form, that is characterized by thermal DSC data, as measured by the DSC method described herein above, substantially identical to those depicted in FIG. 9. C-Type crystals may be obtained, e.g., from evaporations, slow cool, and slurry experiments in methanol (MeOH).

The DSC curve shows three endothermic events at onset temperatures of 111° C., 129° C., and 157° C. when heated at 10° C./min.

The C-type crystal form is a methanol solvate, i.e., methanolate, and TG analysis of the C-type crystals shows a weight loss of approximately 4.1% between 25° C. and 150° C. The weight loss corresponds to approximately 0.5 mol of methanol. Thermogravimetric infrared (TG-IR) analysis is performed to confirm that the weight loss is due to the release of methanol. The TG portion of the analysis shows a 5.0% weight loss between 18° C. and 125° C. IR analysis identified the volatile as methanol. The linked spectrum is taken at the maximum absorption. The waterfall plot shows that the release of methanol gradually increases during the heating process. XRPD analysis of the collected solid material shows similarities to B-type crystals.

Thermogravimetric infrared (TG-IR) analyses may be acquired, e.g., on a TA Instruments thermogravimetric (TG) analyzer model 2050 interfaced to a Magna 560® Fourier transform infrared (FT-IR) spectrophotometer (Thermo Nicolet) equipped with an Ever-Glo mid/far IR source, a potassium bromide (KBr) beamsplitter, and a deuterated triglycine sulfate (DTGS) or MCT detector. The TG instrument is operated under a flow of helium at 90 and 10 cc/min for the purge and balance, respectively. Each sample is placed in an aluminum sample pan, inserted into the TG furnace, accurately weighed by the instrument, and the furnace is heated from ambient to 130 or 150° C. at a rate of 20° C./min. The TG instrument is started first, immediately followed by the FT-IR instrument. Each IR spectrum represents 8 co-added scans collected at a spectral resolution of 4 cm⁻¹. IR spectra are collected approximately every 12 seconds for approximately 8 min. A background scan is collected before the beginning of the experiment. Wavelength calibration is performed using polystyrene. The TG calibration standards are nickel and Alumel™. Volatiles are identified from a search of the High Resolution Nicolet TGA Vapor Phase spectral library.

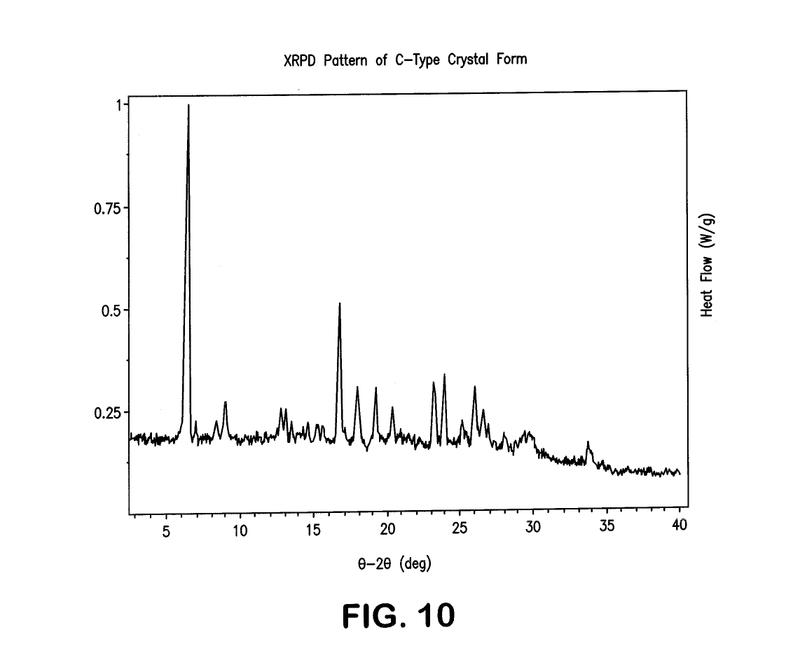

An example of an X-ray diffraction pattern exhibited by C-type crystal form is substantially identical to that depicted in FIG. 10, having characteristic peaks, expressed in degrees 2-theta (2θ), of about 6.3±0.2, 16.7±0.2, 18.0±0.2, 19.2±0.2, 23.2±0.2, 23.9±0.2 and 26.0±0.2. The present invention also provides C-type crystal form that exhibits an X-ray diffraction pattern substantially the same as that depicted in FIG. 10, having characteristic diffraction peaks expressed in degrees 2-theta, and relative intensities at approximately the values shown in Table 3 herein below:

TABLE 3
C-Type Crystal Form of MRE-0094

| Angle (deg 2θ) | Relative Intensity (%) |
|---|---|
| 6.3 ± 0.2 | 100.0 |
| 6.9 ± 0.2 | 22.3 |
| 8.3 ± 0.2 | 22.4 |
| 8.9 ± 0.2 | 27.4 |
| 12.7 ± 0.2 | 25.9 |
| 13.0 ± 0.2 | 25.5 |
| 13.4 ± 0.2 | 22.2 |
| 14.5 ± 0.2 | 22.0 |
| 15.2 ± 0.2 | 21.3 |
| 15.5 ± 0.2 | 21.1 |
| 16.7 ± 0.2 | 51.2 |
| 18.0 ± 0.2 | 30.5 |
| 19.2 ± 0.2 | 30.2 |
| 20.4 ± 0.2 | 25.3 |
| 23.2 ± 0.2 | 31.4 |
| 23.9 ± 0.2 | 33.4 |
| 25.2 ± 0.2 | 22.1 |
| 26.0 ± 0.2 | 30.2 |
| 26.6 ± 0.2 | 24.5 |
| 28.0 ± 0.2 | 19.0 |
| 29.4 ± 0.2 | 19.3 |
| 33.7 ± 0.2 | 16.4 |

Desolvation studies under various conditions may be performed to investigate the effects of drying conditions on the C-type crystals. It appears that the C-type crystals convert to A-type crystal form when heated at 60° C. for eight days or when dried in vacuo for six days. The C-type crystal form does not completely convert to A-type crystals under ambient conditions after eight days of exposure. However, the anhydrous form to which C-type crystals convert may be dependent on the drying conditions and temperature.

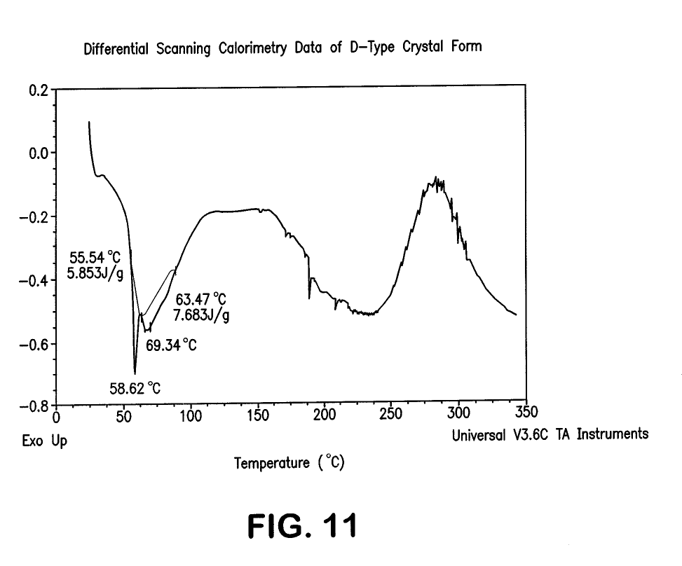

In yet another aspect, the present invention provides a crystal form of MRE-0094, designated herein as D-type crystal form, that is characterized by thermal DSC data, as measured by the DSC method described herein above, substantially identical to those depicted in FIG. 11. D-Type crystals may be obtained, e.g., by slow evaporation from 2,2,2-trifluoroethanol (TFE).

The DSC curve shows two endothermic events at onset temperatures of 56° C. and 63° C. when heated at 10° C./min. TG analysis shows a weight loss of approximately 5% between 25° C. and 200° C. The 5% weight loss corresponds to approximately 0.25 mol of TFE. The D-type crystal form appears to be a TFE solvate.

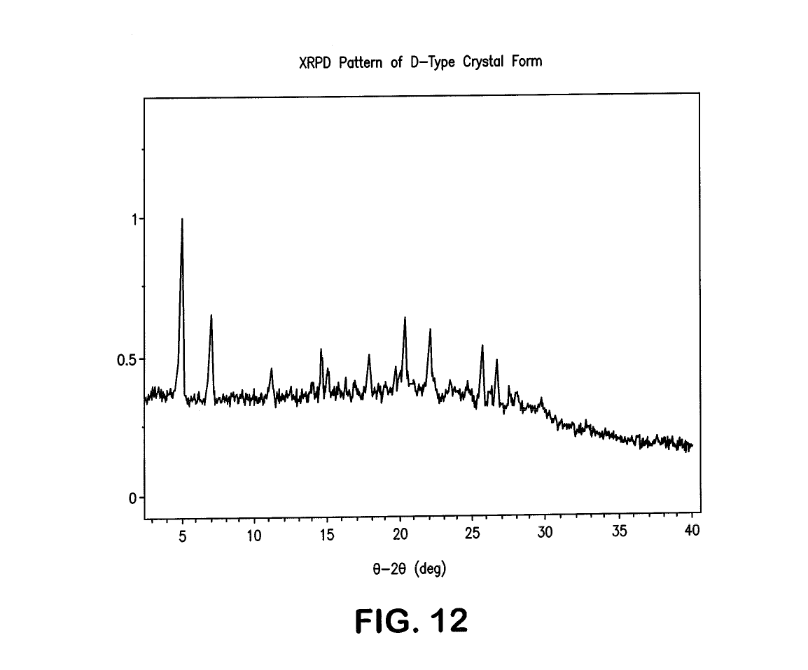

An example of an X-ray diffraction pattern exhibited by D-type crystal form is substantially identical to that depicted in FIG. 12, having characteristic peaks, expressed in degrees 2-theta (2θ), of about 4.9±0.2, 6.9±0.2, 14.5±0.2, 17.8±0.2, 20.3±0.2, 22.0±0.2 and 25.6±0.2. The present invention also provides D-type crystal form that exhibits an X-ray diffraction pattern substantially the same as that depicted in FIG. 12, having characteristic diffraction peaks expressed in degrees 2-theta, and relative intensities at approximately the values shown in Table 4 herein below.

TABLE 4
D-Type Crystal Form of MRE-0094

| Angle (deg 2θ) | Relative Intensity (%) |
|---|---|
| 4.9 ± 0.2 | 100.0 |
| 6.9 ± 0.2 | 65.1 |
| 11.1 ± 0.2 | 46.1 |
| 14.0 ± 0.2 | 40.6 |
| 14.5 ± 0.2 | 52.7 |
| 15.0 ± 0.2 | 45.3 |
| 17.8 ± 0.2 | 50.3 |
| 19.6 ± 0.2 | 45.8 |
| 20.3 ± 0.2 | 63.3 |
| 22.0 ± 0.2 | 59.0 |
| 23.3 ± 0.2 | 41.8 |
| 23.5 ± 0.2 | 40.9 |
| 23.8 ± 0.2 | 38.6 |
| 24.7 ± 0.2 | 40.4 |
| 25.6 ± 0.2 | 53.0 |
| 26.1 ± 0.2 | 36.7 |
| 26.6 ± 0.2 | 47.9 |
| 27.5 ± 0.2 | 38.6 |
| 28.0 ± 0.2 | 36.5 |

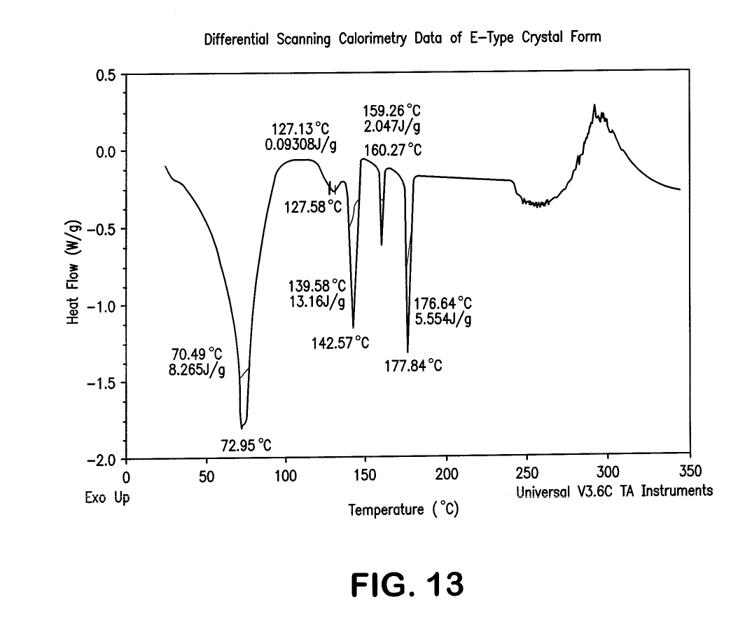

in yet another aspect, the present invention provides a crystal form of MRE-0094, designated herein as E-type crystal form, that is characterized by thermal DSC data, as measured by the DSC method described herein above, substantially identical to those depicted in FIG. 13. E-Type crystals may be obtained, e.g., from a slurry in water.

The DSC analysis of E-type crystals of MRE-0094 shows an endothermic event at an onset temperature of 70° C. when heated at 10° C./min, which may be due to dehydration. Endothermic events are also observed at onset temperatures of 127° C., 140° C., 159° C., and 177° C. TG analysis shows a weight loss of approximately 3.2% between 25° C. and 200° C.

TG-IR analysis may be performed to confirm that the weight loss is due to the release of water. The TG portion of the analysis shows a rapid weight loss of 4.9% between 25° C. and 150° C. The drastic weight loss begins at approximately 25° C., which may indicate that E-type crystal form is an unstable hydrate under the conditions investigated. IR analysis identified the volatile as water. The linked spectrum is taken at the maximum absorption. The waterfall plot shows that the release of water is continuous throughout the heating process. The collected solid material remaining after TG-IR is identified by XRPD as amorphous/low crystalline material.

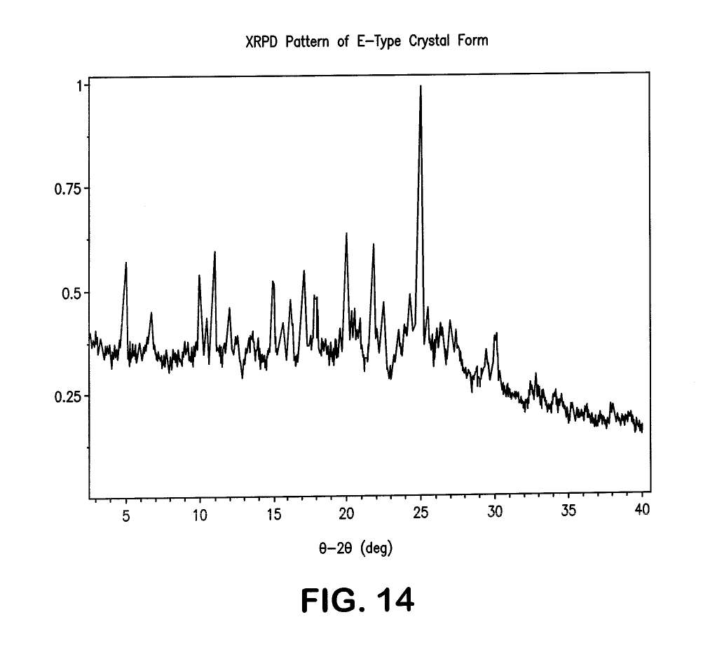

An example of an X-ray diffraction pattern exhibited by E-type crystal form is substantially identical to that depicted in FIG. 14, having characteristic peaks, expressed in degrees 2-theta (2θ), of about 4.9±0.2, 9.9±0.2, 10.8±0.2, 14.9±0.2, 17.1±0.2, 17.8±0.2, 19.9±0.2, 21.8±0.2, 24.2±0.2 and 24.9±0.2. The present invention also provides E-type crystal form that exhibits an X-ray diffraction pattern substantially the same as that depicted in FIG. 14, having characteristic diffraction peaks expressed in degrees 2-theta, and relative intensities at approximately the values shown in Table 5 herein below:

TABLE 5
E-Type Crystal Form of MRE-0094

| Angle (deg 2θ) | Relative Intensity (%) |
|---|---|
| 4.9 ± 0.2 | 58.2 |
| 6.4 ± 0.2 | 41.2 |
| 6.7 ± 0.2 | 46.3 |
| 9.9 ± 0.2 | 55.3 |
| 10.4 ± 0.2 | 45.3 |
| 10.8 ± 0.2 | 60.6 |
| 11.9 ± 0.2 | 47.7 |
| 12.5 ± 0.2 | 40.8 |
| 13.6 ± 0.2 | 42.1 |
| 14.9 ± 0.2 | 53.9 |
| 15.6 ± 0.2 | 43.9 |
| 16.0 ± 0.2 | 49.3 |
| 17.1 ± 0.2 | 56.3 |
| 17.8 ± 0.2 | 50.5 |
| 19.9 ± 0.2 | 64.6 |
| 20.3 ± 0.2 | 46.7 |
| 20.5 ± 0.2 | 47.5 |
| 20.9 ± 0.2 | 44.7 |
| 21.8 ± 0.2 | 62.4 |
| 22.5 ± 0.2 | 48.6 |
| 23.5 ± 0.2 | 42.1 |
| 24.2 ± 0.2 | 50.5 |
| 24.9 ± 0.2 | 100.0 |
| 25.5 ± 0.2 | 47.5 |
| 26.1 ± 0.2 | 42.2 |
| 26.4 ± 0.2 | 43.4 |
| 26.9 ± 0.2 | 43.9 |
| 27.4 ± 0.2 | 41.8 |
| 29.4 ± 0.2 | 37.1 |
| 30.1 ± 0.2 | 40.8 |
| 32.4 ± 0.2 | 29.2 |
| 32.8 ± 0.2 | 31.0 |

Karl Fisher analysis of E-type crystals shows variable water contents of 10%, 13%, and 23%, which corresponds to approximately 2.5, 3.5, and 7 mol of water, respectively.

Coulometric Karl Fischer (KF) analysis for water determination may be performed using, e.g., a Mettler Toledo DL38 Karl Fischer titrator with the Mettler Toledo D0307 drying oven attachment. The sample is placed in the KF drying oven and dried allowing the water vapor to leave the solid material and enter the titration vessel containing approximately 150 mL of Hydranal-Coulomat AD. The water vapor from the sample is then titrated by means of a generator electrode which produces iodine by electrochemical oxidation: 2 I⁻=>I₂+2e. Three replicates are obtained to ensure reproducibility.

The E-type crystal form of MRE-0094 appears to be a crystalline hydrate that may be unstable when exposed to ambient or drying conditions.

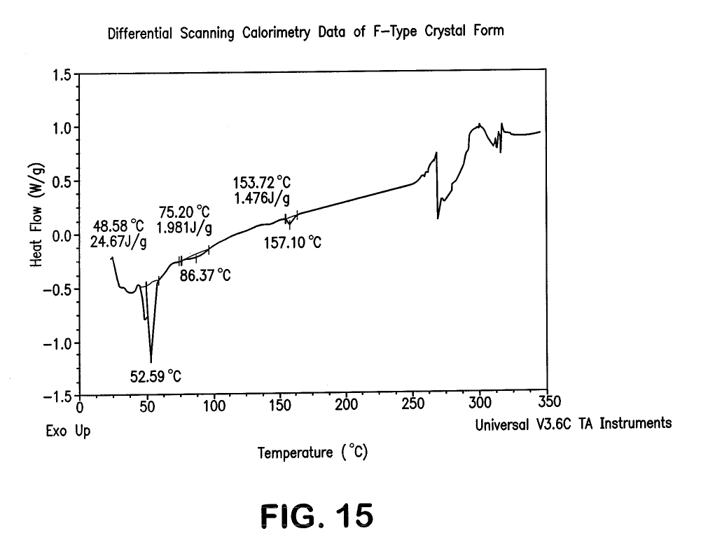

In yet another aspect, the present invention provides a crystal form of MRE-0094, designated herein as F-type crystal form, that is characterized by thermal DSC data, as measured by the DSC method described herein above, substantially identical to those depicted in FIG. 15. F-Type crystals may be obtained, e.g., by slow cool and slurry experiments in EtOH.

The DSC analysis of F-type crystal form shows three endothermic events with onset temperatures of 49° C., 75° C., and 154° C. when heated at 10° C./min.

TG analysis shows a weight loss of approximately 2.6% between 25° C. and 150° C. The weight loss is not accurate since the TG data shows a weight loss before the analysis began. A weight loss of 2.6% corresponds to approximately 0.25 mol of ethanol.

TG-IR analysis may be performed to confirm that the weight loss is due to the release of ethanol. The TG portion of the analysis shows a two step weight loss with 5.3% of weight lost between 18° C. and 80° C. followed by an additional 0.6% weight loss between 80° C. and 125° C. IR analysis identified the volatiles as ethanol and water. The linked spectrum is taken at the maximum absorption. The waterfall plot shows that water is released initially followed by the gradual release of ethanol during the heating process. It appears that both water and ethanol are associated within the crystal structure. XRPD analysis of the solid material remaining after the TG-IR analysis is identified as a low crystalline mixture of A-type and B-type crystals.

Desolvation studies under various conditions may be conducted to investigate the effects of drying conditions on F-type crystals. Exposure to vacuum oven and 60° C. results in a low crystalline mixture of A-type and B-type crystals. Amorphous/low crystalline material is obtained by exposing F-type crystals to ambient conditions for eight days.

The F-type crystal form appears to be an ethanol solvate, i.e., ethanolate, with associated water. This solid material appears to readily desolvate upon exposure to drying conditions.

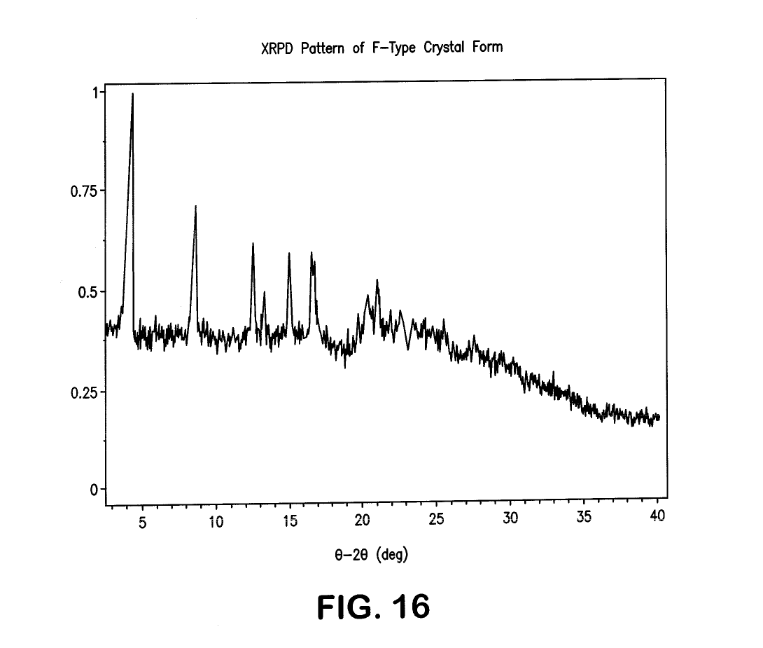

An example of an X-ray diffraction pattern exhibited by F-type crystal form is substantially identical to that depicted in FIG. 16, having characteristic peaks, expressed in degrees 2-theta (2θ), of about 4.0±0.2, 8.4±0.2, 12.3±0.2, 14.8±0.2, 16.3±0.2, 16.5±0.2 and 20.9±0.2. The present invention also provides F-type crystal form that exhibits an X-ray diffraction pattern substantially the same as that depicted in FIG. 16, having characteristic diffraction peaks expressed in degrees 2-theta, and relative intensities at approximately the values shown in Table 6 herein below

TABLE 6
F-Type Crystal Form of MRE-0094

| Angle (deg 2θ) | Relative Intensity (%) |
|---|---|
| 4.0 ± 0.2 | 100.0 |
| 8.4 ± 0.2 | 71.3 |
| 12.3 ± 0.2 | 61.9 |
| 12.9 ± 0.2 | 44.2 |
| 13.1 ± 0.2 | 49.7 |
| 13.4 ± 0.2 | 41.8 |
| 14.8 ± 0.2 | 59.4 |
| 16.3 ± 0.2 | 58.8 |
| 16.5 ± 0.2 | 57.5 |
| 16.7 ± 0.2 | 44.5 |
| 19.5 ± 0.2 | 44.2 |
| 20.2 ± 0.2 | 48.7 |
| 20.5 ± 0.2 | 46.0 |
| 20.9 ± 0.2 | 52.5 |
| 21.7 ± 0.2 | 44.6 |
| 22.4 ± 0.2 | 44.9 |
| 23.3 ± 0.2 | 42.7 |
| 24.1 ± 0.2 | 42.9 |
| 24.6 ± 0.2 | 40.7 |
| 25.3 ± 0.2 | 42.5 |
| 26.0 ± 0.2 | 36.9 |
| 27.4 ± 0.2 | 38.5 |

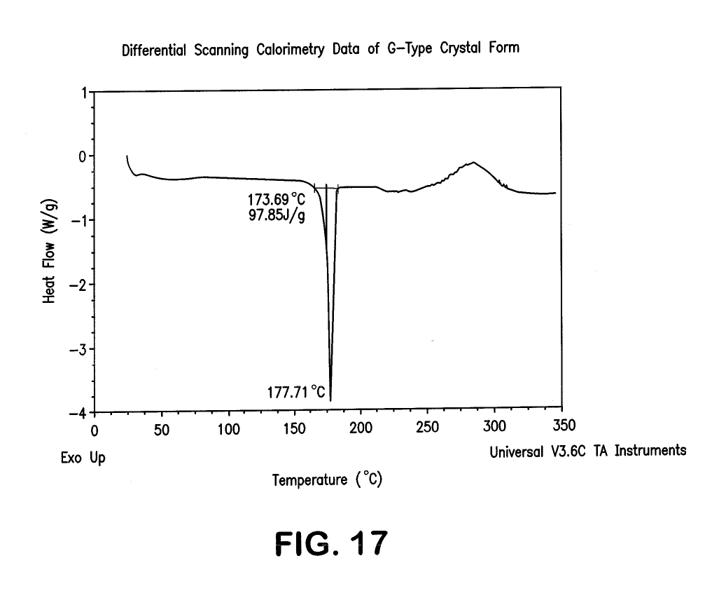

Yet in another aspect, the present invention provides a crystal form of anhydrous MRE-0094, designated herein as G-type crystal form, having a melting point of about 178° C. as measured by the DSC method described herein above, and as depicted in FIG. 17. G-Type crystals may be obtained by a variety of techniques, e.g., from a slurry in isopropanol (IPA).

The DSC analysis of G-type crystal form shows only one endotherm with onset and maximum temperatures of 174° C. and 178° C., respectively, when heated at 10° C./min. TG analysis shows a 0.5% weight loss between 25° C. and 200° C. Significant weight loss is observed after 200° C., which may be due to decomposition.

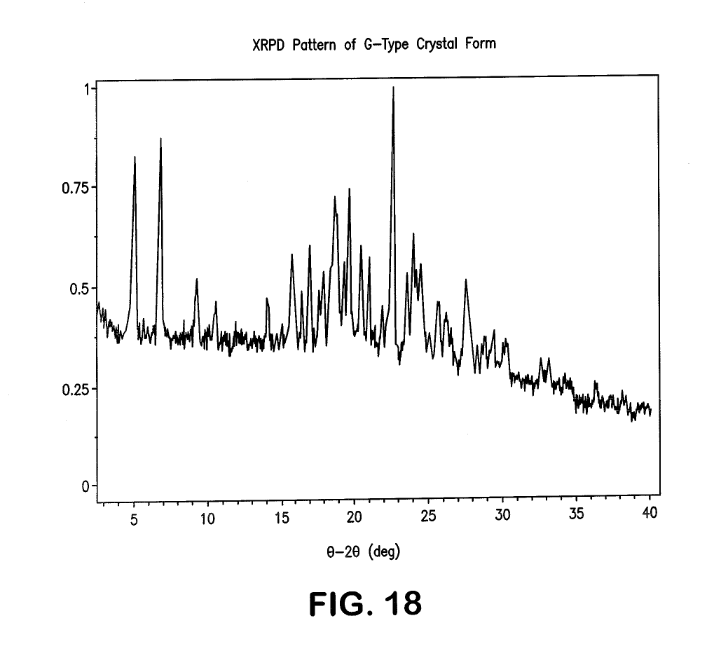

An example of an X-ray diffraction pattern exhibited by G-type crystal form is substantially identical to that depicted in FIG. 18, having characteristic peaks, expressed in degrees 2-theta (2θ), of about 4.9±0.2, 6.7±0.2, 16.9±0.2, 18.5±0.2, 19.5±0.2, 20.3±0.2, 22.5±0.2 and 23.9±0.2. The present invention also provides G-type crystal form that exhibits an X-ray diffraction pattern substantially the same as that depicted in FIG. 18, having characteristic diffraction peaks expressed in degrees 2-theta, and relative intensities at approximately the values shown in Table 7 herein below:

TABLE 7
G-Type Crystal Form of MRE-0094

| Angle (deg 2θ) | Relative Intensity (%) |
|---|---|
| 4.9 ± 0.2 | 83.2 |
| 6.7 ± 0.2 | 87.3 |
| 9.1 ± 0.2 | 52.3 |
| 9.9 ± 0.2 | 41.0 |
| 10.4 ± 0.2 | 46.9 |
| 11.0 ± 0.2 | 39.2 |
| 13.9 ± 0.2 | 47.2 |
| 15.6 ± 0.2 | 58.1 |
| 16.2 ± 0.2 | 48.9 |
| 16.9 ± 0.2 | 60.5 |
| 17.4 ± 0.2 | 49.0 |
| 17.8 ± 0.2 | 53.8 |
| 18.5 ± 0.2 | 72.9 |
| 19.2 ± 0.2 | 56.1 |
| 19.5 ± 0.2 | 74.5 |
| 20.3 ± 0.2 | 60.2 |
| 20.9 ± 0.2 | 57.1 |
| 21.8 ± 0.2 | 45.2 |
| 22.5 ± 0.2 | 100.0 |
| 23.5 ± 0.2 | 53.3 |
| 23.9 ± 0.2 | 63.0 |
| 24.4 ± 0.2 | 55.2 |
| 25.0 ± 0.2 | 37.9 |
| 25.6 ± 0.2 | 45.9 |
| 26.1 ± 0.2 | 41.2 |
| 26.2 ± 0.2 | 42.3 |
| 27.5 ± 0.2 | 51.5 |
| 28.3 ± 0.2 | 34.6 |
| 28.8 ± 0.2 | 36.8 |
| 29.3 ± 0.2 | 38.7 |
| 29.9 ± 0.2 | 35.4 |
| 30.2 ± 0.2 | 35.8 |
| 32.5 ± 0.2 | 31.5 |
| 33.0 ± 0.2 | 31.5 |
| 36.2 ± 0.2 | 25.4 |

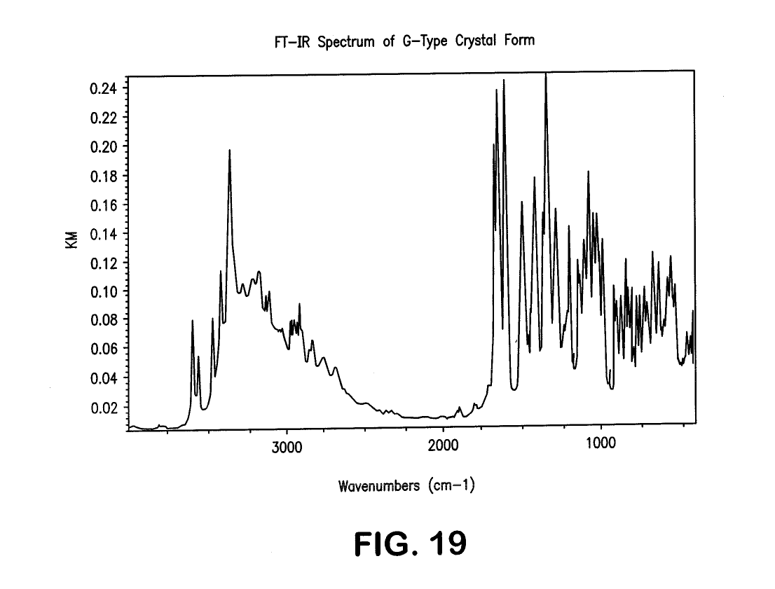

An example of an infrared absorption spectrum of G-type crystal form obtained by the KBr method is shown in FIG. 19, and is characterized by absorption bands at about 3470±2, 3250-3100, 3050-3020, 2980-2820, 2785-2750, 2350-2315, 1920-1875 and 1800-1780 cm⁻¹.

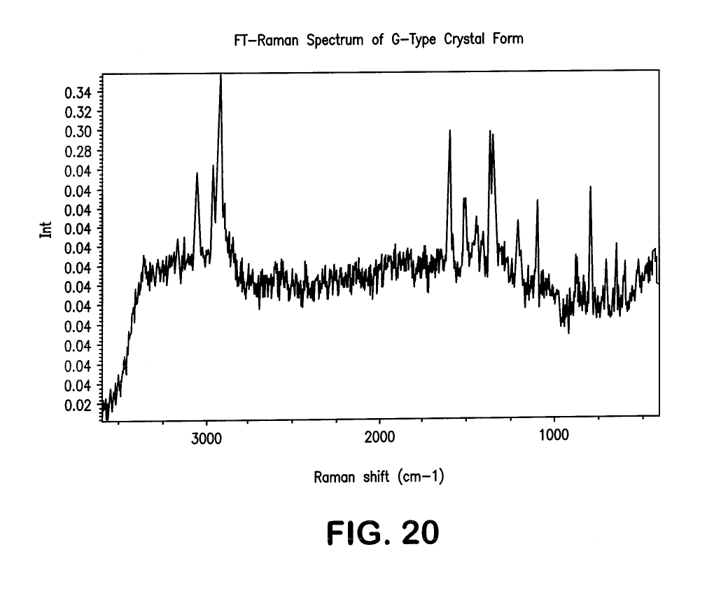

An example of a FT-Raman spectrum of G-type crystal form obtained by the method described herein above is shown in FIG. 20, and is characterized by Raman shifts at about 3050±2, 2970±2, 1600±2, 1510±2 (doublet), 1360-1345 (doublet) and 1310±2 cm⁻¹.

The G-type crystal form of MRE-0094 appears to be anhydrous, and moisture sorption analysis shows a total weight gain of approximately 2.4% (approximately 0.5 mol of water) between 5% and 95% RH. Approximately 2.2% of the weight is lost upon desorption. The solid material remaining after moisture sorption analysis is identified as the G-type crystal form by XRPD. Based on the moisture sorption data, the G-type crystal form appears to be a hygroscopic solid when exposed to high relative humidity.

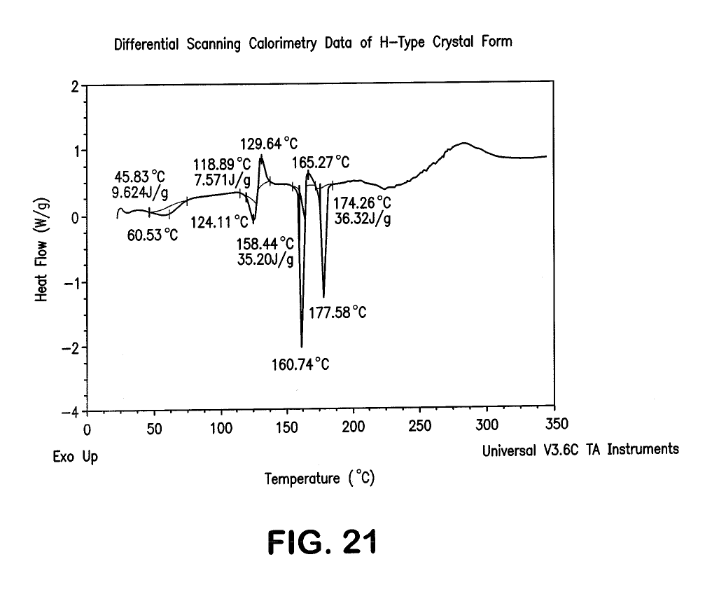

In yet another aspect, the present invention provides a crystal form of MRE-0094, designated herein as H-type crystal form, that is characterized by thermal DSC data, as measured by the DSC method described herein above, substantially identical to those depicted in FIG. 21. H-Type crystals may be obtained, e.g., by stressing the A-type crystal form at 68% or 75% relative humidity.

The DSC analysis of H-type crystal form shows a broad endotherm at 61° C. when heated at 10° C./min that is likely due to dehydration. The remaining thermal events are consistent with the thermal events observed for the A-type crystal form. TG analysis shows a negligible weight loss of approximately 0.4% between 25° C. and 150° C.

Further examination of the XRPD pattern suggests that this material may be a mixture of A-type and I-type crystals, or A-type crystals displaying additional diffraction peaks at 14.5±0.2 and 20.4±0.2° 2θ with minor peak shifting.

This solid material does not appear to be a unique crystalline phase, and is likely a mixture of A-type and I-type crystals, or solely A-type crystal form.

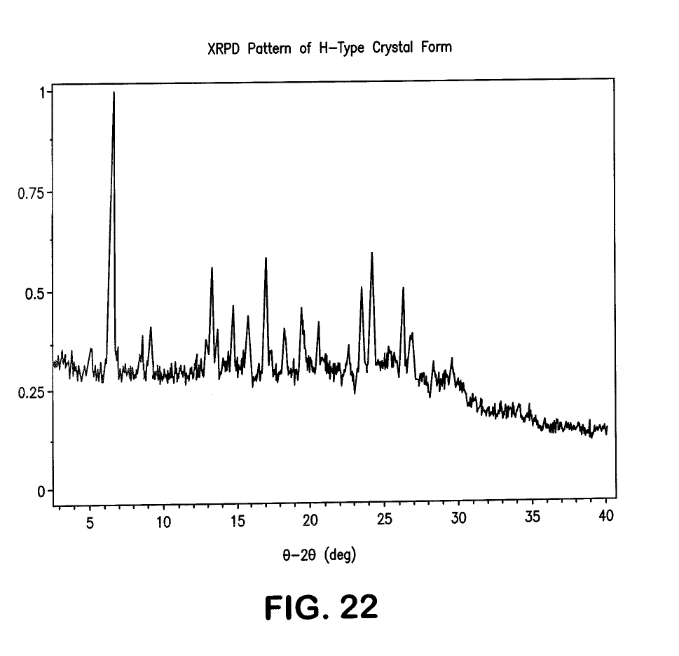

An example of an X-ray diffraction pattern exhibited by H-type crystals is substantially identical to that depicted in FIG. 22

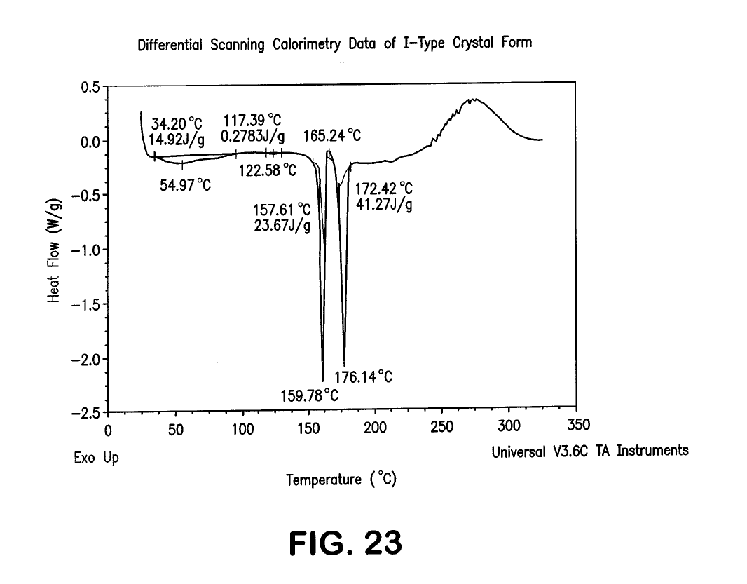

In yet another aspect, the present invention provides a crystal form of MRE-0094, designated herein as I-type crystal form, that is characterized by thermal DSC data, as measured by the DSC method described herein above, substantially identical to those depicted in FIG. 23. I-Type crystals may be obtained, e.g., by stressing the A-type crystal form at 97% relative humidity for 3-14 days.

The DSC analysis of I-type crystal form shows a broad endotherm at 55° C. when heated at 10° C./min, which may be due to dehydration. Other thermal events are observed at onset temperatures of 117° C. (endothermic), 158° C. (endothermic), 165° C. (exothermic), and 172° C. (endothermic) TG analysis shows a negligible weight loss of approximately 0.6% between 25° C. and 150° C. Karl Fisher analysis indicates that the solid material contains approximately 6.2% of water, which corresponds to approximately 1.5 mol of water.

TG-IR analysis may be performed to better understand this solid material and to confirm that the weight loss is due to the release of water. The TG portion of the analysis shows a rapid 3.1% weight loss between 18° C. and 125° C. The weight loss begins immediately at ambient conditions, which may suggest that I-type crystal form is an unstable hydrate under these conditions. IR analysis identifies the volatile as water. The linked spectrum is taken at the maximum absorption. The waterfall plot shows that the release of water is continuous throughout the heating process. The collected solid material remaining after TG-IR is identified by XRPD as the B-type crystal form.

Dehydration studies under various conditions are performed to investigate the effects of drying conditions on the I-type crystals. In each case, the solid material consists of either B-type, or a mixture of A-type and B-type crystals after dehydration.

The I-type crystal form appears to be a crystalline hydrate that may contain approximately 1.5 mol of water.

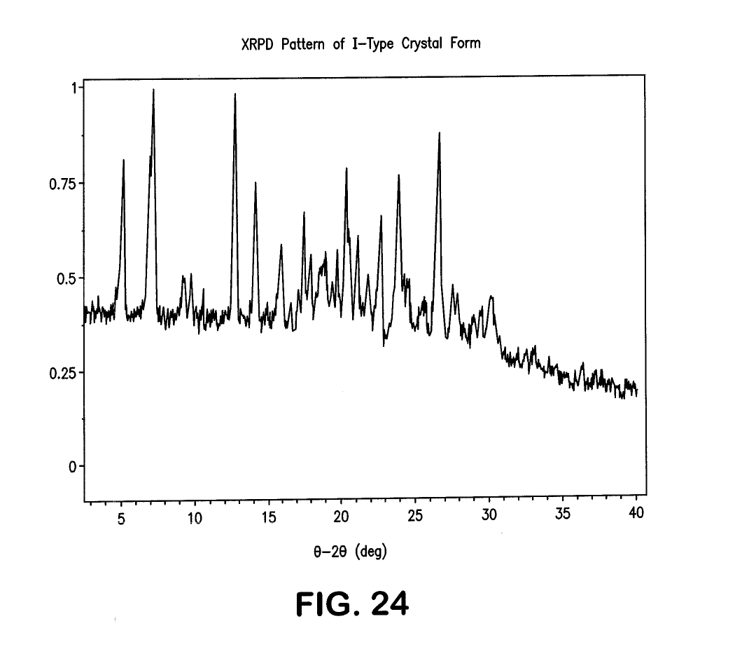

An example of an X-ray diffraction pattern exhibited by I-type crystal form is substantially identical to that depicted in FIG. 24, having characteristic peaks, expressed in degrees 2-theta (2θ), of about 4.9±0.2, 6.7±0.2, 6.9±0.2, 13.9±0.2, 17.2±0.2, 20.1±0.2, 22.5±0.2, 23.8±0.2 and 26.5±0.2. The present invention also provides I-type crystal form that exhibits an X-ray diffraction pattern substantially the same as that depicted in FIG. 24, having characteristic diffraction peaks expressed in degrees 2-theta, and relative intensities at approximately the values shown in Table 8 herein below:

TABLE 8
I-Type Crystal Form of MRE-0094

| Angle (deg 2θ) | Relative Intensity (%) |
|---|---|
| 4.9 ± 0.2 | 81.3 |
| 6.7 ± 0.2 | 84.5 |
| 6.9 ± 0.2 | 100.0 |
| 9.0 ± 0.2 | 50.3 |
| 9.5 ± 0.2 | 50.8 |
| 12.0 ± 0.2 | 42.2 |
| 13.9 ± 0.2 | 75.0 |
| 15.6 ± 0.2 | 58.6 |
| 16.4 ± 0.2 | 43.1 |
| 16.8 ± 0.2 | 46.7 |
| 17.2 ± 0.2 | 67.2 |
| 17.7 ± 0.2 | 55.7 |
| 18.1 ± 0.2 | 46.0 |
| 18.4 ± 0.2 | 52.9 |
| 18.7 ± 0.2 | 56.7 |
| 19.2 ± 0.2 | 48.8 |
| 19.5 ± 0.2 | 57.2 |
| 20.1 ± 0.2 | 78.8 |
| 20.3 ± 0.2 | 62.9 |
| 21.0 ± 0.2 | 60.7 |
| 21.7 ± 0.2 | 50.6 |
| 22.5 ± 0.2 | 66.0 |
| 23.8 ± 0.2 | 76.6 |
| 24.1 ± 0.2 | 50.6 |
| 24.3 ± 0.2 | 49.2 |
| 24.5 ± 0.2 | 47.0 |
| 25.1 ± 0.2 | 41.8 |
| 25.3 ± 0.2 | 42.0 |
| 25.5 ± 0.2 | 44.4 |
| 25.6 ± 0.2 | 42.6 |
| 26.5 ± 0.2 | 87.9 |
| 27.5 ± 0.2 | 47.7 |
| 27.7 ± 0.2 | 44.9 |
| 28.1 ± 0.2 | 37.3 |
| 28.8 ± 0.2 | 39.5 |
| 29.2 ± 0.2 | 36.8 |
| 30.0 ± 0.2 | 44.4 |
| 32.4 ± 0.2 | 30.2 |
| 33.0 ± 0.2 | 30.9 |

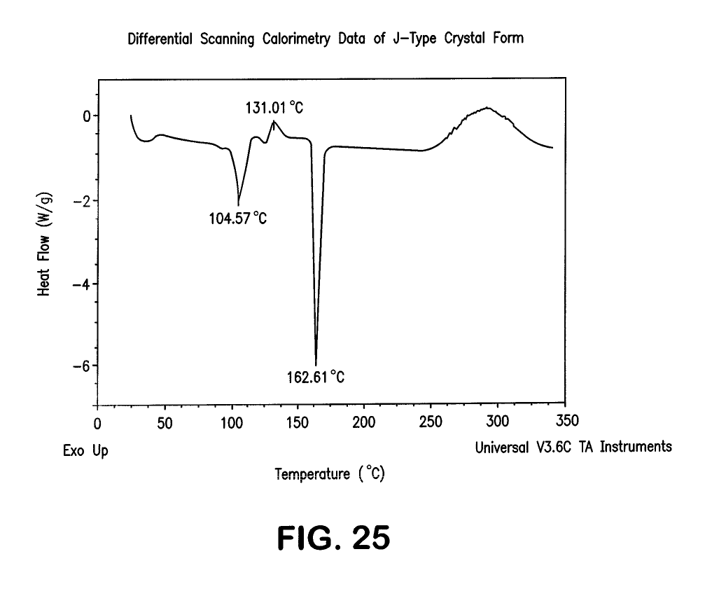

In yet another aspect, the present invention provides a crystal form of MRE-0094, designated herein as J-type crystal form, that is characterized by thermal DSC data, as measured by the DSC method described herein above in an open pan, substantially identical to those depicted in FIG. 25. J-Type crystal form is an ethanol solvate, and may be obtained, e.g., from a slurry in EtOH.

The DSC analysis of J-type crystal form shows a broad endotherm at about 105° C. when heated at 20° C./min that is likely due to desolvation. The remaining thermal events are a small exotherm at about 131° C., and a second endotherm at about 163° C.

The TG portion of the TG-IR analysis shows a weight loss of approximately 67% between about 22° C. and about 150° C. The linked IR spectrum identifies the volatile as ethanol. Progressive increase (maximal signal at about 5.8 min) and decrease of signal correlates to one step weight loss in time seen on TG analysis.

The J-type crystal form appears to be a crystalline ethanol solvate that may contain approximately from 0.5 to 1 mol of EtOH.

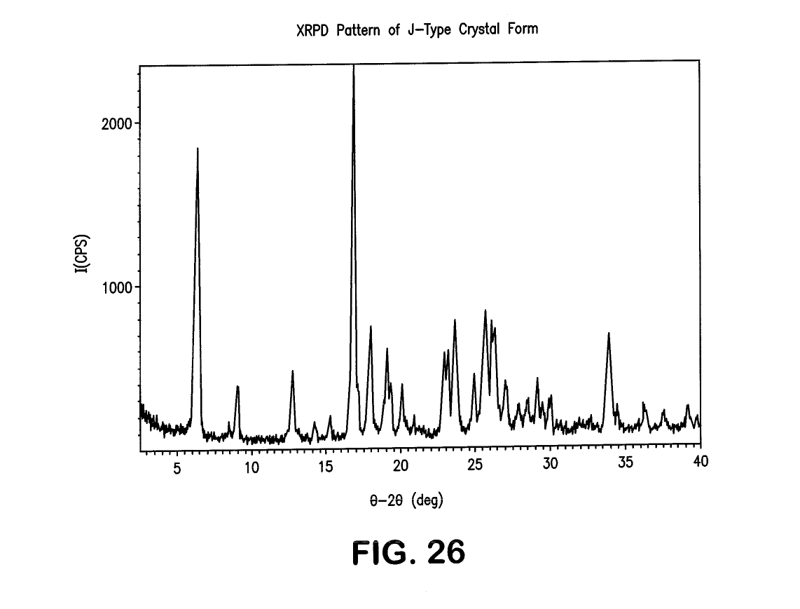

An example of an X-ray diffraction pattern exhibited by J-type crystal form is substantially identical to that depicted in FIG. 26, having characteristic peaks, expressed in degrees 2-theta (2θ), of about 6.4±0.2, 15.2±0.2, 16.8±0.2, 23.6±0.2, 25.7±0.2, 26.1±0.2 and 26.3±0.2. The present invention also provides J-type crystal form that exhibits an X-ray diffraction pattern substantially the same as that depicted in FIG. 26, having characteristic diffraction peaks expressed in degrees 2-theta, and relative intensities at approximately the values shown in Table 9 herein below:

TABLE 9
J-Type Crystal Form of MRE-0094

| Angle (deg 2θ) | Relative Intensity (%) |
|---|---|
| 6.4 ± 0.2 | 67 |
| 9.0 ± 0.2 | 12 |
| 12.7 ± 0.2 | 22 |
| 15.2 ± 0.2 | 34 |
| 16.8 ± 0.2 | 100 |
| 17.9 ± 0.2 | 26 |
| 19.0 ± 0.2 | 21 |
| 19.3 ± 0.2 | 16 |
| 20.1 ± 0.2 | 13 |
| 23.0 ± 0.2 | 28 |
| 23.2 ± 0.2 | 27 |
| 23.6 ± 0.2 | 37 |
| 24.9 ± 0.2 | 14 |
| 25.7 ± 0.2 | 35 |
| 26.1 ± 0.2 | 30 |
| 26.3 ± 0.2 | 30 |
| 27.1 ± 0.2 | 12 |
| 27.8 ± 0.2 | 6 |
| 29.1 ± 0.2 | 10 |
| 30.0 ± 0.2 | 8 |
| 34.0 ± 0.2 | 29 |
| 34.2 ± 0.2 | 12 |

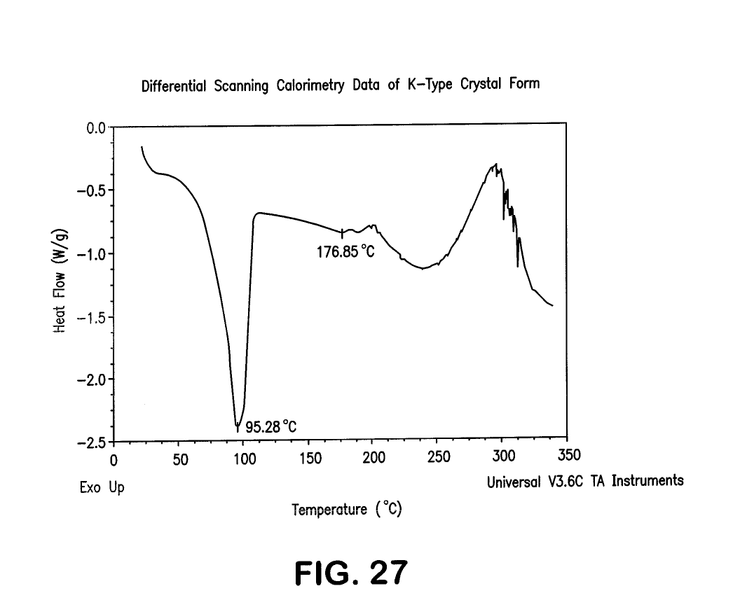

In yet another aspect, the present invention provides a crystal form of MRE-0094, designated herein as K-type crystal form, that is characterized by thermal DSC data, as measured by the DSC method described herein above in an open pan, substantially identical to those depicted in FIG. 27. K-Type crystal form is an IPA solvate of MRE-0094, and may be obtained, e.g., from a slurry of F-type crystals in a mixture of IPA and heptane.

The DSC analysis of K-type crystal form shows a broad endotherm at about 95° C. when heated at 20° C./min that is likely due to desolvation, and possibly melting. The remaining thermal events are a very small endotherm at about 177° C. followed by decomposition.

The TG portion of the TG-IR analysis shows a weight loss of approximately 8.4% between about 22° C. and about 120° C. The linked IR spectrum identifies the volatile as IPA. Progressive increase (maximal signal at about 5.4 min) and decrease of signal correlates to one step weight loss in time seen on TG analysis.

The K-type crystal form appears to be a crystalline IPA solvate that may contain approximately from 0.5 to 1 mol of IPA.

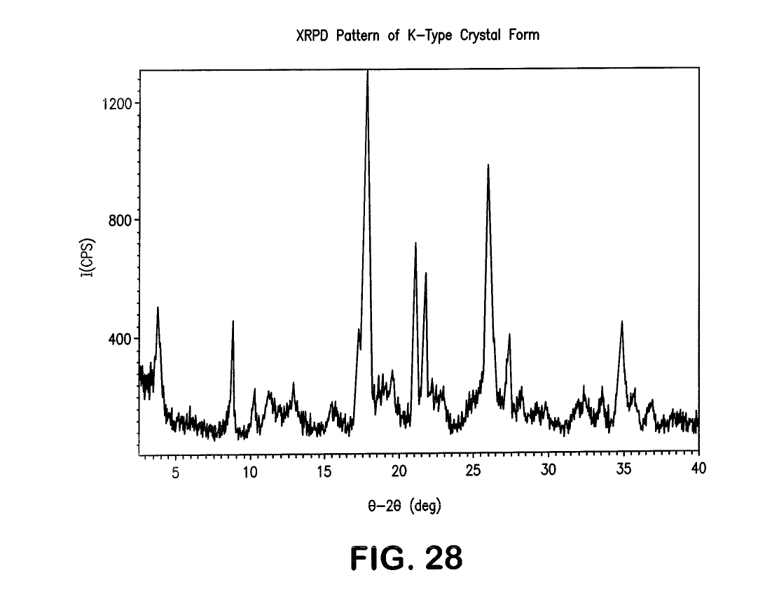

An example of an X-ray diffraction pattern exhibited by K-type crystal form is substantially identical to that depicted in FIG. 28, having characteristic peaks, expressed in degrees 2-theta (20), of about 17.6±0.2, 21.0±0.2, 21.6±0.2 and 26.3±0.2. The present invention also provides J-type crystal form that exhibits an X-ray diffraction pattern substantially the same as that depicted in FIG. 26, having characteristic diffraction peaks expressed in degrees 2-theta, and relative intensities at approximately the values shown in Table 10 herein below:

TABLE 10
K-Type Crystal Form of MRE-0094

| Angle (deg 2θ) | Relative Intensity (%) |
|---|---|
| 3.8 | 27 |
| 8.7 | 27 |
| 10.1 | 9 |
| 11.2 | 9 |
| 12.7 | 9 |
| 15.6 | 7 |
| 17.6 | 100 |
| 18.8 | 10 |
| 19.5 | 14 |
| 21.0 | 51 |
| 21.6 | 40 |
| 22.8 | 9 |
| 22.1 | 11 |
| 25.8 | 70 |
| 26.3 | 20 |
| 27.2 | 21 |
| 28.0 | 6 |
| 32.2 | 8 |
| 33.5 | 8 |
| 34.7 | 28 |
| 35.6 | 9 |

Preferred are the crystal forms of MRE-0094 that are substantially stable to grinding. Stability to grinding may be assessed by measurement of an appropriate physical property before and after grinding. Where the physical property remains substantially unchanged substantial stability to grinding is indicated. Suitable physico-chemical properties for measurement include a melting point or DSC analysis, a X-ray diffraction pattern, an infrared absorption spectrum and a Raman spectrum, particularly the X-ray diffraction pattern. For example, the G-type crystal form of MRE-0094 is substantially stable to mechanical milling followed by exposure of the milled sample to about 97% relative humidity (vapor stress, K₂SO₄ humidity jar, 1 day) as indicated by XRPD analyses of the milled and vapor stressed material, respectively.

Stability to mechanical grinding/milling may be tested, e.g., by employing a small stainless steel cylinder The cylinder is charged with a weighted amount of a crystal form of MRE-0094 and a stainless steel ball is added, the cylinder is capped and the unit is placed on a Retsch Mixer Mill (Type MM 200) for a total of 30 min at a frequency of 30/s (ball-milling).

In a further aspect, the present invention provides methods for the production of different crystal forms of MRE-0094 wherein the method comprises dissolving MRE-0094, in any of its forms, including amorphous forms, in a suitable solvent, including mixed solvents, forming the MRE-0094 crystals, isolating and drying the crystal form of MRE-0094, including hydrates and solvates, e.g., methanol and ethanol solvates.

The dissolution and crystallization may be carried out in several ways as will be apparent to those of ordinary skill in the art.

In one embodiment of the present invention, crystal forms of MRE-0094 may be produced by dissolving of MRE-0094, in any of its forms, in a solvent to form a solution at a dissolution temperature ranging from room temperature (RT) to the boiling point of the solvent, in which solvent MRE-0094 is suitably soluble at that temperature, and in which solvent MRE-0094 is only poorly soluble at a lower temperature ranging from about −20° C. to RT, cooling the solution to the lower temperature to induce precipitation of a crystal form of MRE-0094, isolating and drying the precipitated crystal form of MRE-0094, in particular the A-type, C-type, F-type, G-type and J-type crystal forms of MRE-0094.

Solvents in which MRE-0094 is suitably soluble at a temperature ranging from RT to the boiling point of the solvent, and in which MRE-0094 is only poorly soluble at a lower temperature ranging from about −20° C. to RT include, but are not limited to, lower alcohols such as MeOH, EtOH and IPA, and the like. Preferred mixed solvents include a mixture of a polar solvent such as a lower alcohol, e.g., methanol and ethanol, preferably ethanol, with water. When a mixed solvent is employed, preferably a mixture of methanol or ethanol with water, more preferably a mixture of ethanol with water, the concentration of the polar solvent in the solvent mixture is generally about 50% by volume or more. The dissolution temperature preferably ranges from about 50° C. to about 80° C., and more preferably from about 60° C. to about 75° C. The amount of MRE-0094 in the solvent ranges preferably from about 0.5% to about 50% by weight of the resulting mixture, more preferably from about 3% to about 30% by weight. If the amount of MRE-0094 is more than 50% by weight then the slurry properties of the initial suspension are poor and it will be difficult to agitate the mixture and properly dissolve the solid. On the other hand, it is not efficient in terms of the volume of the solvent required to use less than 0.5% of MRE-0094 by weight. The lower temperature to which the solution of MRE-0094 is cooled to induce precipitation of the desired crystal form of MRE-0094 ranges from about −20° C. to RT, preferably the lower temperature is RT. It should be noted that the rate in which the solution is cooled to the lower temperature may also affect which crystal form of MRE-0094 is produced. Furthermore, it may be advantageous to add seed crystals of the desired crystal form to the solution to further aid precipitation. The resulting mixture may then be maintained at the lower temperature for a time sufficient to assure complete precipitation of the desired form of MRE-0094 crystals.

In a specific embodiment, the solvent in which MRE-0094 is suitably soluble is IPA. A solution of MRE-0094, e.g., A-type crystals of MRE-0094, is generated by dissolution at about 60° C. in an oil bath set up on a hotplate. The solution is then allowed to cool to RT in the oil bath, heaters switched off (slow cool=SC). The precipitated crystals are isolated by vacuum filtration to give G-type crystals of MRE-0094. Additional examples are shown in Table 11.

TABLE 11

| Solvent | Conditions | XRPD Result |
|---|---|---|
| EtOH | 60° C., SC | F-type crystals |
| EtOH | 66° C., FC^{a} | J-type crystals |
| EtOH | 75° C., SC | J-type crystals |
| EtOH:H₂O - 1:1 | 60° C., SC | G-type crystals |
| MeOH | 60° C., SC | C-type crystals |

^{a}FC = fast cool, i.e., the solution is taken out of the oil bath and allowed to cool to RT

Alternatively, crystal forms of MRE-0094 can be produced by a method wherein the method comprises dissolving MRE-0094, in any of its forms including amorphous form, in a solvent, referred to herein as a first solvent (S), to form a solution, in which solvent MRE-0094 is readily soluble at a temperature ranging from RT to the boiling point of the solvent, treating the solution with another solvent, referred to herein as a second solvent (an anti-solvent, AS), which is miscible with the first solvent and in which MRE-0094 is only poorly soluble, to induce precipitation of a crystal form of MRE-0094, isolating and drying the precipitated crystal form of MRE-0094, in particular the E-type and G-type crystal forms of MRE-0094.

First solvents in which MRE-0094 is readily soluble, i.e., in amounts of at least 3% by weight at the dissolution temperature, include, but are not limited to, polar solvents such as IPA, propylene glycol (PG) and tetrahydrofuran (THF). Second solvents in which MRE-0094 is only poorly soluble, i.e., in amounts of 0.5% by weight or less, include put are not limited to, water and heptane. The dissolution temperature, i.e., the temperature in which MRE-0094 is dissolved in the first solvent, ranges preferably from RT to about the boiling point of the solvent. In a preferred embodiment of the present invention, the first solvent is IPA and the dissolution temperature is about 85° C. The amount of MRE-0094 dissolved in the first solvent ranges preferably from 1 to 50% by weight of the resulting solution. The solution of MRE-0094 in the first solvent may be added to the second solvent, or the second solvent may be added to the solution of MRE-0094 in the first solvent. It may be advantageous to filter the solution of MRE-0094 in the first solvent prior to induction of precipitation. The ratio of the first solvent to the second solvent in the resulting mixture ranges preferably from about 1:1 to about 1:9 by volume, more preferably from about 1:1 to about 1:2 by volume. It may also be advantageous to include seed crystals in the mixture to aid precipitation of the desired MRE-0094 crystals. The seed crystals may be added the solution of MRE-0094 in the first solvent, or they may be included in the second solvent prior to the combination of the second solvent with the solution of MRE-0094 in the first solvent. The solution of MRE-0094 in the first solvent may also be cooled a lower temperature ranging from RT to about 50° C. prior to mixing. Alternatively, the second solvent may be cooled to a lower temperature ranging from about −20° C. to RT prior to mixing. The resulting mixture containing the desired crystal form of MRE-0094 may then be maintained at the lower temperature for a time sufficient to assure complete precipitation of the desired MRE-0094 crystals.

In a specific embodiment, the first solvent (S) is THF and the second solvent (AS) is water. MRE-0094, e.g., A-type crystals of MRE-0094, is dissolved in the first solvent at RT, followed by filtration of the resulting solution through a 0.2-μm nylon filter into excess amount of the second solvent at ambient temperature. The precipitated crystals are then collected by vacuum filtration to give E-type crystals of MRE-0094

In another embodiment, the first solvent (S) is IPA and the second solvent (AS) is heptane. MRE-0094, in any of its forms, is dissolved in IPA at 85° C., and G-type seed crystals are added to the solution. The mixture is then cooled gradually to 10° C. to induce precipitation, followed by addition of the second solvent. The precipitated crystals are then collected by vacuum filtration and dried to give G-type crystals of MRE-0094. Additional examples are shown in Table 12.

TABLE 12

| S/AS | Conditions | XRPD Result |
|---|---|---|
| THF:heptane | 57° C.^{a} | G-type crystals |
| PG:water | RT^{b} | G-type crystals |
| PG:water | RT^{c} | E-type crystals |

^{a}Second solvent added to the solution of MRE-0094 in the first solvent, then cooled to RT.
^{b}The solution of MRE-0094 in the first solvent is added to the second solvent.
^{c}Second solvent added to the solution of MRE-0094 in the first solvent.

Alternatively to cooling and/or addition of anti-solvent to induce precipitation, a solution of MRE-0094, e.g., any one of those described herein above, may be concentrated by evaporation to certain fraction of the original volume to induce precipitation of the desired crystal form of MRE-0094 Preferably, the solution of MRE-0094 is evaporated to dryness at an ambient temperature. It should be noted that the rate in which the solution is concentrated may also affect which crystal form of MRE-0094 is produced, i.e., fast evaporation (FE) may be carried out without covering the vessel containing the solution of MRE-0094, or said vessel may be covered with a foil perforated with pinholes to affect slow evaporation (SE).

In a specific embodiment, MRE-0094, e.g., A-type crystals of MRE-0094, is treated with aliquots of MeOH at RT to form a solution. The resulting solution of MRE-0094 is then filtered through a 0.2-μm nylon filter to remove any remaining solids, and the filtrate is allowed to evaporate to dryness at RT without cover (FE) to produce C-type crystals of MRE-0094. Additional examples are shown in Table 13.

TABLE 13

| Solvent | Conditions | XRPD Result |
|---|---|---|
| MeOH | RT; SE | C-type crystals |
| THF | RT; FE | G-type crystals |
| THF | RT; SE | B-type crystals |
| TFE | RT; SE | D-type crystals |

In an alternative aspect of the present invention, solid MRE-0094 is suspended in a suitable solvent at a temperature of at least 10° C. and up to the boiling point of the solvent wherein MRE-0094 is incompletely soluble, preferably only sparingly soluble, in the solvent at that temperature. A suspension/slurry results in which particles of solid are dispersed, and remain incompletely dissolved in the solvent. Preferably, the solids are maintained in a state of suspension by agitation, e.g., by shaking or stirring. The suspension/slurry is then kept at a temperature of 10° C. or higher for a time sufficient to effect transformation of the starting solids into product crystals. The product crystals may then be isolated and dried using conventional methods in the art.

The solid MRE-0094 to be suspended in a suitable solvent may be in any of its forms, e.g., the A-type crystal form, or it may be a solvate, e.g., hydrate, methanolate, ethanolate or isopropanolate. Preferably, the suspension is maintained at said temperature of at least 10° C. for a time sufficiently long so that the product crystals contain the desired crystal form of MRE-0094 substantially free of any alternate polymorphic forms of MRE-0094. Furthermore, the time for which the suspension is maintained at said temperature of at least 10° C. (slurry time) varies depending on the nature of the solvent(s) used, the temperature and other factors, such as the quantity of solids in the suspension and the size of the solid particles. Generally, however, the slurry time may be in the range of from about 10 min to 15 days. By adding seed crystals of the desired crystal form of MRE-0094 to the dispersion, the time required to form said crystals of MRE-0094 may be shortened. The end point in formation of the desired crystals of MRE-0094 may be determined by sampling crystals from the suspension, for example by filtration during the course of the conversion followed by measuring, e.g., the powder X-ray diffraction pattern of the crystals.

Solvents suitable for use in this embodiment of the present invention include water, esters, such as methyl acetate and EtOAc, lower alcohols, such as MeOH, EtOH, IPA and TFE, ketones, such as acetone and methylethylketone (MEK), and solvents such as t-2butylmethylether (TBME), DCM, THF, dioxane, acetonitrile (MeCN), PG and heptane, or a suitable mixture of miscible solvents thereof, e.g., water mixed with a lower alcohol, preferably with EtOH, or heptane or acetone mixed with IPA. The temperature of the suspension preferably ranges from about 10° C. to about 50° C. Preferably, the amount of MRE-0094 dispersed in the solvent is from 0.5% to 50% by weight of the resulting suspension. If the amount of MRE-0094 dispersed is more than 50% by weight then the slurry properties of the suspension are poor and it will be difficult to agitate. On the other hand, it is not efficient in terms of the volume of solvent required to use less than 0.5% by weight. Preferably the suspension includes from 5% to 15% of solids by weight.

In a specific embodiment, EtOAc is saturated at RT with A-type crystals of MRE-0094 (at about 2 g scale) while stirring, so that solids persist. The slurry is then stirred for an additional day at RT. The resulting solids are collected by vacuum filtration and dried in vacuo at ambient temperature to afford B-type crystals of MRE-0094. Additional examples using A-type crystals as the starting material are shown in Table 14.

TABLE 14

| Solvent | Temperature | Slurry time | XRPD Result |
|---|---|---|---|
| EtOAc | 42° C. | 1 to 2 days | B-type crystals |
| TBME | RT | 1 day | A-type crystals |
| acetone | RT | 2 h to 1 day | G-type crystals |
| acetone | 42° C. | 2 days | G-type crystals |
| MeCN | RT | 6 days | G-type crystals |
| DCM | RT | 14 days | B-type crystals |
| dioxane | RT | 9 days | G-type crystals |
| THF | RT | 1 to 9 days | G-type crystals |
| heptane | RT | 14 days | A-type crystals |
| MEK | RT | 9 days | B-type crystals |
| TFE | RT | 9 days | G-type crystals |
| PG | RT | 11 days | G-type crystals |
| MeOH | RT | 9 days | C-type crystals |
| EtOH | RT | 9 days | F-type crystals |
| IPA | RT | 10 min to 14 days | G-type crystals |
| water | RT | 2 to 14 days | E-type crystals |
| EtOH:water - 1:1 | RT | 15 days | G-type crystals |
| IPA:acetone - 1:1 | RT | 2 h | G-type crystals |
| IPA:heptane - 1:1^{a} | 10° C. | 1 h | G-type crystals |

^{a}IPA added first, immediately followed by heptane.

Generally, the use of a mixed solvent gives rise to favorable results if the G-type crystal form is to be produced, e.g., a solvent which is a mixture of IPA and heptane is particularly effective. The final concentration of IPA in the solvent mixture ranging from about 25% to 75% (by volume) is preferred. More preferably, the concentration of IPA in the solvent mixture is about 50% The temperature of the suspension preferably ranges from about 10° C. to about 50° C. It should be noted that a suspension of MRE-0094 in a mixture of solvents may be formed sequentially, i.e., MRE-0094 may be first suspended to a first solvent (S1), then agitated, cooled and/or warmed prior to the addition of a second solvent (S2). As the data in Table 15 illustrates, G-type crystals are obtained reproducibly in about 90-96% yield from slurry experiments employing A-type crystals as the starting material (2 or 6 g scale) and a mixture of IPA and heptane as the solvent. When a slurry in IPA as a sole solvent is employed, G-type crystals are obtained in about 81-91% yield.

TABLE 15

| Solvent | Slurry conditions | | XRPD Result, Yield (%) |
|---|---|---|---|
| IPA | IPA (29.8 mL), 23° C., stir for 1 h | | G-type crystals^{a}, 81 |
| IPA | IPA (20 mL), 15° C., stir for 2 h | | G-type crystals^{a}, 84 |
| IPA | IPA (19.8 mL), 23° C., stir for 3 h | | G-type crystals^{a}, 88 |
| IPA | IPA (15 mL), 23° C., stir for 15 min | | G-type crystals^{a}, 84 |
| IPA | IPA (15 mL), 23° C., stir for 10 min | | G-type crystals^{a}, 91 |
| IPA | 1. | IPA (29.8 mL), 20° C., warm to 40° C. | |
| | | in 30 min | |
| | 2. | 40° C. for 60 min | G-type crystals^{a}, 84 |
| | 3. | 40° C. to 20° C. in 60 min | |
| | 4. | 20° C. for 30 min | |
| IPA | 1. | IPA (29.8 mL), 50° C., stir for 30 min | G-type crystals^{a}, 86 |
| | 2. | 50° C. to 20° C. in 140 min | |
| IPA (S1) | 1. | S1 (9.9 mL), 10° C., immediately | G-type crystals^{a}, 92 |
| heptane (S2) | | followed by S2 (9.9 mL) | |
| | 2. | stir at 10° C. for 1 h | |
| IPA:heptane - 2:1 (S1) | 1. | S1 (14.8 mL), 23° C., stir for 30 min | G-type crystals^{a}, 94 |
| heptane (S2) | 2. | S2 (10 mL) added (over 2 min) | |
| | 3. | stir at 23° C. for 30 min | |
| IPA (S1) | 1. | S1 (15 mL), 20° C., stir for 1 h | G-type crystals^{a}, 91 |
| heptane (S2) | 2. | S2 (15 mL) added, 0.5 mL/min | |
| | | (over 30 min), 20° C. | |
| | 3. | stir at 20° C. for 90 min | |
| IPA (S1) | 1. | S1 (15 mL), 23° C., stir for 30 min | G-type crystals^{a}, 94 |
| heptane (S2) | 2. | S2 (15 mL) added over a period of | |
| | | about 15 min, 23° C. | |
| | 3. | stir at 23° C. for 16 min | |
| IPA (S1) | 1. | S1 (45 mL), 20° C., stir for 30 min | G-type crystals^{b}, 95 |
| heptane (S2) | 2. | S2 added (45 mL) over a period of | |
| | | about 10 min, 20° C. | |
| | 3. | 20° C. to 10° C. in 50 min (0.2° C./min) | |
| | 4. | stir at 10° C. for 40 min | |
| IPA (S1) | 1. | S1 (40.2 mL), 10° C., stir for 30 min | G-type crystals^{b}, 95 |
| heptane (S2) | 2. | S2 (60 mL) added over a period of | |
| | | about 15 min, 10° C. | |
| | 3. | stir at 10° C. for 1 h 15 min | |
| IPA:heptane - 2:1 (S1) | 1. | S1 (45 mL), 23° C., stir for 10 min | G-type crystals^{b}, 96 |
| heptane (S2) | 2. | S2 (30 mL) added over a period of | |
| | | 10 min, 23° C. | |
| | 3. | stir at 23° C. for 1 h 40 min | |
| IPA (S1) | 1. | S1 (45 mL), 23° C., stir for 30 min | G-type crystals^{b}, 96 |
| heptane (S2) | 2. | S2 (45 mL) added over a period of | |
| | | about 15 min, 23° C. | |
| | 3. | stir at 23° C. for 30 min | |
| IPA (S1) | 1. | S1 (45 mL), 15° C., stir for 30 min | G-type crystals^{b}, 96 |
| heptane (S2) | 2. | S2 (45 mL) added over a period of | |
| | | about 15 min, 15° C. | |
| | 3. | stir at 15° C. for 30 min | |
| IPA (S1) | 1. | S1 (45 mL), 30° C., stir for 30 min | G-type crystals^{b}, 92 |
| heptane (S2) | 2. | S2 (45 mL) added over a period of | |
| | | about 15 min, 30° C. | |
| | 3. | stir at 30° C. for 30 min | |
| IPA (S1) | 1. | S1 (45 mL), 20° C., stir for 30 min | G-type crystals^{b}, 95 |
| heptane (S2) | 2. | S2 (45 mL) added over a period of | |
| | | about 15 min, 20° C. | |
| | 3. | stir at 20° C. for 30 min | |
| IPA (S1) | 1. | S1 (36 mL), 23° C., stir for 30 min | G-type crystals^{b}, 95 |
| heptane (S2) | 2. | S2 (36 mL) added over a period of | |
| | | about 15 min, 23° C. | |
| | 3. | stir at 23° C. for 30 min | |
| IPA (S1) | 1. | S1 (54 mL), 23° C., stir for 30 min | G-type crystals^{b}, 94 |
| heptane (S2) | 2. | S2 (54 mL) added over a period of | |
| | | about 15 min, 23° C. | |
| | 3. | stir at 23° C. for 30 min | |
| IPA (S1) | 1. | S1 (45 mL), 23° C., stir for 30 min | G-type crystals^{b}, 95 |
| heptane (S2) | 2. | S2 (45 mL) added over a period of | |
| | | about 15 min, 23° C. | |
| | 3. | stir at 23° C. for 3 h | |
| IPA (S1) | 1. | S1 (15 mL), 20° C., stir for 30 min | G-type crystals^{a,c}, >81^{e} |
| heptane (S2) | 2. | S2 (15 mL) added over a period of | |
| | | about 15 min, 20° C. | |
| | 3. | stir at 15° C. for 30 min | |
| IPA (S1) | 1. | S1 (14.8 mL), 15° C., stir for 30 min | K-type crystals^{a,d}, >46^{e} |
| heptane (S2) | 2. | S2 (14.8 mL) added over a period | |
| | | of about 15 min, 20° C. | |
| | 3. | stir at 20° C. for 30 min | |

^{a}2 g scale.
^{b}6 g scale.
^{c}J-Type crystals are employed as the starting material.
^{d}F-Type crystals are employed as the starting material.
^{e}Yields are given as “greater than” as the starting material is not dried prior to being weighed.

Accordingly, the present invention provides a use of the A-type crystal form for the manufacture of the G-type crystal form.

As described herein above, conventional methods, such as heating, agitation by sonication and stirring, may be used to aid dissolution of MRE-0094. MRE-0094, in any of its crystal forms, may be added to the solvent or the solvent may be added onto MRE-0094, stirred and optionally heated up to the boiling point of the solvent to form a solution. Stirring, cooling and addition of seed crystals may be used to further induce precipitation of the desired crystal form of MRE-0094. The precipitated crystals may be isolated by conventional methods, such as vacuum filtration or centrifugation. The crystals may be washed, preferably with a solvent or a solvent mixture consisting of solvents used in the crystallization. During isolation and washing, cooling may be applied, if so desired. The isolated MRE-0094 crystals may be dried under atmospheric or reduced pressure, preferably under reduced pressure ranging from about 20 mmHg to about 0.1 mmHg, and at a temperature ranging from RT to about 50° C.

According to a still further aspect of the present invention, herein is provided a method for the preparation of a pharmaceutical composition, preferably a topical composition, for promoting wound healing comprising the use of a therapeutically effective amount of a crystal form of MRE-0094, as obtainable by the methods of the present invention, in particular G-type crystals of MRE-0094, and pharmaceutically acceptable excipients, diluents or carriers thereof.

Likewise, the present invention provides use of a crystal form of MRE-0094, as obtainable by the methods of the present invention, in particular G-type crystals of MRE-0094, for the preparation of a pharmaceutical composition, preferably a topical composition, for promoting wound healing.

The term “therapeutically effective amount” as used herein refers to an amount of MRE-0094 that will elicit the desired biological or medical response of a tissue, system or a mammal (including man) that is being sought by a researcher or clinician, in particular a therapeutically effective amount refers to an amount of MRE-0094 effective to promote wound healing, e.g., to promote healing of diabetic foot ulcers.

The term “wound healing” shall be understood herein to include the expressions “promoting wound healing” and “accelerating wound healing”, and refers to treatment of all wound types, acute or chronic, such that the wound undergoes healing and closure more rapidly than similar wounds left to heal naturally. Preferably, wound healing and closure takes place without systemic absorption of the active ingredient.

The term “mammal, warm-blooded animal or patient” are used interchangeably herein and include, but are not limited to, humans, dogs, cats, horses, pigs, cows, monkeys, rabbits, mice and laboratory animals. The preferred mammals are humans.

The term “treatment” shall be understood as the management and care of a patient for the purpose of combating the disease, condition or disorder, e.g., accelerating healing of a wound.

As described herein above, crystal forms of MRE-0094 may be employed in the manufacture of pharmaceutical compositions comprising a therapeutically effective amount of MRE-0094 in a drug delivery vehicle useful in the treatment of wounds Preferably, MRE-0094 is administered via topical route, e.g., the pharmaceutical composition is a gel, a cream, an ointment or a lotion Preferably, the pharmaceutical composition is a gel.

The pharmaceutical compositions comprising MRE-0094 may include about 10% to about 70% w/w glycol, preferably about 20% to about 60% w/w glycol, most preferably about 50% w/w glycol. In particular, the glycol may be a C₁-C₉ alkyl diol or the polymer of the diol. Preferably, the glycol is propylene glycol.

The pharmaceutical compositions may further comprise a thickening agent Examples of suitable thickening agents include, but are not limited to, acacia, alginic acid, bentonite, carbomer, carboxymethylcellulose sodium, cetostearyl alcohol, colloidal silicon dioxide, ethylcellulose, gelatin, guar gum, hydroxyethyl-cellulose, hydroxypropyl cellulose, hydroxypropyl methylcellulose, magnesium aluminum silicate, maltodextrin, methylcellulose, polyvinyl alcohol, povidone, propylene carbonate, propylene glycol alginate, sodium alginate, sodium starch glycolate, starch, tragacanth, and xanthan gum. In an embodiment of the subject invention, the thickening agent is a microcrystalline cellulose. Preferably, the microcrystalline cellulose is sodium carboxymethylcellulose.

Preferably, the pharmaceutical compositions comprising MRE-0094 are administered topically once a day. Preferably, the patient is administered an amount of MRE-0094 ranging from about 0.1 μg/day to about 1000 μg/day. A sterile applicator swab may be used to apply a thin, uniform film (approximately the thickness of a dime) of the topical composition comprising MRE-0094 in a concentration ranging from about 0.5 μg/g to about 500 μg/g over the entire surface area of the wound. The wound may then be covered with an appropriate dressing.

The efficacy of MRE-0094 in promoting wound healing may be assessed, e.g., as follows:

### A. Phase I Study Design

MRE-0094 is administered to patients with neuropathic, diabetic foot ulcers (DFU). Patients 18-80 years old are randomized in a 1:3 ratio to a standard DFU care and a vehicle gel, or a standard DFU care and a gel containing MRE-0094 as described herein above. Standard care include routine sharp debridement, pressure offloading, and maintaining a moist wound environment. Inclusion criteria includes full thickness wounds between 1 and 10 cm² in area. Exclusion criteria includes arterial insufficiency, renal or hepatic insufficiency, active infection, or osteomyelitis. Patients are enrolled into 3 groups, and receive drug at an escalating concentration by group of 5 μg/g, 50 μg/g, or 500 μg/g. Safety data are reviewed by an independent safety board prior to escalating dose. Drug or vehicle is applied topically once daily for 28 days. Outcome measures include adverse events and other safety assessments, plasma concentrations of the active agent MRE-0094, percent of wound closure, and rate of wound closure.

Thirty-six patients are randomized (25 active agent, 11 vehicle gel, i.e., placebo) with an average age of 54.8 years, 78% are male. No systemic absorption of MRE-0094 is detected at any topical dose concentration The mean (±SD) wound size at randomization determined by planimetry is 0.91±0.63 cm² and does not differ between vehicle and MRE-0094 groups. Percent wound closure at 28 days, and median days to 50% and 75% closure by treatment groups are listed in Table 16

### B. Phase II Study Design

This study is a multicenter, double-blind, randomized, parallel, vehicle-controlled, and standard care-controlled trial of topically applied MRE-0094 in diabetic subjects with chronic, neuropathic foot ulcers. A broad range of concentrations of the invention are studied in a wide variety of wound sizes. Approximately 340 subjects are enrolled in these studies. Efficacy endpoints include the incidence of complete healing (complete epithelialization with no exudate) of the wounds, time to wound closure (days), and percent reduction in surface area of the wounds from baseline to various time points after start of the MRE-0094 treatment. Safety assessments include evaluating systemic exposure to topical MRE-0094 measured by plasma concentrations of MRE-0094; adverse events, irritation scores, and other safety parameters routinely monitored in clinical trials. In general, subjects complete a 14-day screening/standard care run-in period, a treatment period of up to 90 days, and a 28-day post-treatment period All subjects receive standard care treatment for their wound(s) throughout the studies that is consistent with the American Diabetes Association Consensus Development Conference on Diabetic Wound Care (American Diabetes Association, Consensus development conference on diabetic foot wound care, Diabetes Care 22(8): 1354-1360, 1999), and the clinical practice guidelines for diabetic foot disorders of the American College of Foot and Ankle Surgeons and the American College of Foot and Ankle Orthopedics and Medicine (Frykberg et al., J Foot Ankle Surg. 39(Suppl 5): S1-60, 2000).

TABLE 16

| | | | Median Number of Days to: | |
|---|---|---|---|---|
| Treatment | N | % Wound Closure (Day 28) | 50% Closure | 75% Closure |
| Vehicle | 11 | 33.3% | 22 | 37 |
| 5 μg/g | 7 | 60.7% | 8 | 14 |
| 50 μg/g | 9 | 67.5% | 12 | 28 |
| 500 μg/g | 9 | 35.4% | 14 | 36 |

The present invention is further described by the following examples. The examples are provided solely to illustrate the invention by reference to specific embodiments. These examples, while illustrating certain specific aspects of the invention, do not portray the limitations or circumscribe the scope of the present invention.

### Example 1

### A Process for Preparing A-Type Crystal Form of MRE-0094

MRE-0094 (crude material as obtained, e.g., by the processes disclosed in U.S. Pat. No. 6,951,932 and U.S. Patent Application Publication No. US 20060135466; pre-crushed using a mortar and pestle, 1973.5 grams) is charged into an appropriately sized 4-neck reactor, equipped with a variable speed overhead stirrer, reflux condenser, nitrogen inlet, positive pressure bubbler, and thermocouple. To the reactor is added 9,868 mL of ethanol, U.S.P., and the mixture is heated to reflux (78±5° C.) until all solids are dissolved. The solution is allowed to cool to 17±5° C. overnight, at which point the crystalline mass is collected by filtration, washed with cold ethanol, and dried under vacuum at 50±5° C. for 12 h. The material is passed through an IKA mill to remove large clumps, then further dried at 50±5° C. under vacuum until the change in weight is less than 0.5% between sequential weightings at least 12 hours apart, providing about 1139 grams of the A-type crystals of MRE-0094.

### Example 2

### A Process for Preparing B-Type Crystal Form of MRE-0094

A. A saturated solution of MRE-0094 (A-type crystal form) containing excess of solids is prepared in an appropriate solvent, e.g., DCM, EtOAc, and MEK The slurry is agitated at RT for 6 to 14 days. Insoluble solids are collected by filtration and allowed to air dry to afford B-type crystals of MRE-0094

B. A solution of MRE-0094 (A-type crystal form) in an appropriate solvent, e.g., EtOH and THF, is prepared and then filtered through a 0.2-μm filter. The solution is left to evaporate slowly in a vial covered with aluminum foil containing pinholes to afford B-type crystals of MRE-0094

C. A sample of MRE-0094 (A-type crystal form) is heated isothermally at approximately 164° C. for 2 min The sample is then removed from the heat source and allowed to cool to RT to afford B-type crystals of MRE-0094

### Example 3

### A Process for Preparing C-Type Crystal Form of MRE-0094

A. A solution of MRE-0094 (A-type crystal form) in MeOH is prepared and filtered through a 0.2-μm filter. The solution is left in an open vial to evaporate quickly at RT or in a vial covered with aluminum foil containing pinholes to evaporate slowly to afford C-type crystals of MRE-0094.

B. A sample of MRE-0094 (A-type crystal form) is dissolved in MeOH on a heated hotplate set at 60° C. The sample is rapidly filtered into a vial kept on the same hotplate. The heat source is then turned off and the hotplate and vial are allowed to cool to RT. The resulting solution is then placed in a refrigerator to cool. Solids formed are collected by decantation of the supernatant and allowed to air dry to afford C-type crystals of MRE-0094.

C. A saturated solution of MRE-0094 (A-type crystal form) containing excess of solids is prepared in MeOH The slurry is agitated at RT for 6 to 14 days. Insoluble solids are collected by filtration and allowed to air dry to afford C-type crystals of MRE-0094.

### Example 4

### A Process for Preparing D-Type Crystal Form of MRE-0094

A solution of MRE-0094 (A-type crystal form) in TFE is prepared and filtered through a 0.2-μm filter. The solution is left in a vial covered with aluminum foil containing pinholes to evaporate slowly at RT to afford D-type crystals of MRE-0094.

### Example 5

### A Process for Preparing E-Type Crystal Form of MRE-0094

A. A saturated solution of MRE-0094 (A-type crystal form) containing excess of solids is prepared in water. The slurry is agitated at RT for 2 days. Solids are recovered by centrifugation followed by decantation of the supernatant and vacuum filtration. Solids are allowed to air dry to afford E-type crystals of MRE-0094.

B. A sample of MRE-0094 (A-type crystal form) is dissolved in PG at RT. Water is added to the filtered solution, causing solids to precipitate. Solids are collected using vacuum filtration, then allowed to air dry to afford E-type crystals of MRE-0094.

### Example 6

### A Process for Preparing F-Type Crystal Form of MRE-0094

A. A sample of MRE-0094 (A-type crystal form) is dissolved in EtOH on a heated hotplate set at 60° C. The sample is rapidly filtered into a vial kept on the same hotplate. The heat source is then turned off and the hotplate and vial are allowed to cool to RT. The resulting solution is then placed in a refrigerator to cool. Solids formed are collected by decantation of the supernatant, and allowed to air dry to afford F-type crystals of MRE-0094.

B. A saturated solution of MRE-0094 (A-type crystal form) containing excess of solids is prepared in EtOH. The slurry is agitated at RT for 9 days. Insoluble solids are recovered via filtration and allowed to air dry to afford F-type crystals of MRE-0094.

### Example 7

### A Process for Preparing G-Type Crystal Form of MRE-0094

A. 6 g of MRE-0094 (A-type crystal form) are placed in a round bottom flask, fitted with a single blade paddle adapted to the contour of the flask and shaft, coupled with a variable speed overhead stirrer, and 45 mL of IPA are added at RT (23° C.) The resulting slurry is then stirred (300 rpm) for 30 min at RT (23° C.), followed by addition of 45 mL of heptane over a period of about 15 min, and the stirring is continued for 3 h further. The solids are collected by vacuum filtration, and the flask and the filter cake are rinsed with 45 mL of heptane. The solids are then dried in a vacuum oven at RT for one day to afford G-type crystals of MRE-0094 (95%).

B. To 840 g of MRE-0094 (crude material as obtained, e.g., by the processes disclosed in U.S. Pat. No. 6,951,932 and U.S Patent Application Publication No. US 20060135466; pre-crushed using a mortar and pestle) in a rotary evaporator bulb is added ethanol (2,500 mL). Using the rotary evaporator, the mixture is heated to reflux to obtain a homogeneous solution, then cool to 16° C. over 12 hours The resulting solids are collected by filtration, then washed with n-heptane (1,700 mL) and allowed to air dry for a minimum of 1 h to afford crude MRE-0094.

Without seed crystals: The collected solids are transferred to a 4-neck, round bottom flask, equipped with a variable speed overhead stirrer, single blade paddle and stir shaft, reflux condenser, temperature probe, nitrogen inlet, bubbler, and chart recorder. To the flask, is added IPA (15,000 ml). The mixture is then heated to 80±5° C. with stirring to dissolve the solids, then cooled using the following controlled cooling profile:

1) the mixture is cooled at approximately 0.1° C./min to 50° C.,
2) the mixture is maintained at 50° C. for 4 h further with stirring; and
3) the mixture is cooled at approximately 0.1° C./minute to 10±5° C.

To the cooled solution, n-heptane (30,250 mL) is added at such a rate that the temperature is maintained at 10±5° C. After completion of the addition, the mixture is stirred for 2 h while continuing to maintain the temperature of the mixture at 10±5° C. The crystallized MRE-0094 is collected by filtration, washed with additional n-heptane (30,250 mL), then dried under vacuum at 50±5° C. to constant weight (less than 0.5% change in mass between sequential weightings at 12 h intervals) Type G crystals are obtained at about 79.5% yield, which is uncorrected for impurities present in the crude MRE-0094.

With seed crystals: 25 g of crude MRE-0094 (pre-crushed using a mortar and pestle) is transferred to a 4-neck 2,000 mL round bottom flask. The flask is placed in a water bath pre-set at 85° C. Pre-warmed IPA (357 mL) is added. The reaction flask is set with a condenser and overhead stirrer (teflon paddle) set at 150 rpm. All solids dissolve within approximately 10 min, and then 250.2 mg of G-type crystals are added to the solution (seed load ˜1 wt %, and are observed to remain undissolved in the hot solution), and the following controlled cooling profile is started;

1) the mixture is cooled at 0.1° C./min from 85° C. to 50° C.;
2) the mixture is maintained at 50° C. for 4 h further; and
3) the mixture is cooled at 0.1° C./min from 50° C. to 10° C.

The mixture is maintained at 10° C. for ˜3 h further, and 714 mL of heptane are added (single addition, final ratio of IPA:heptane is 1:2 v/v). After about 2 h and 20 min, the resulting slurry is removed from the water bath and the solids are collected by vacuum filtration on a paper filter. The filter cake is washed with 714 mL of heptane, dried overnight under vacuum at approximately 50° C., weighed, then dried for additional ˜6.5 h and re-weighed, which showed negligible additional weight loss (˜0.01%), G-Type crystals are obtained at about 86% yield, which is uncorrected for impurities present in the crude material.

### Example 8

### A Process for Preparing I-Type Crystal Form of MRE-0094

A sample of MRE-0094 (A-type crystal form) is placed in a humidity jar at 97% RH at RT for 3 or 14 days to afford I-type crystals of MRE-0094.

### Example 9

### A Process for Preparing J-Type Crystal Form of MRE-0094

A. A solution in EtOH is generated by dissolution of MRE-0094 (A-type crystal form) at about 66° C. in an oil bath set up on a hotplate. The sample is taken out of the oil bath and allowed to cool to RT while stirring and covered with aluminum foil pierced with large holes to allow partial evaporation of the solvent during cooling. The solids are recovered after 3 days by vacuum filtration to afford J-type crystals of MRE-0094

B. A solution in EtOH is generated by dissolution of MRE-0094 (A-type crystal form) at about 75° C. in an oil bath set up on a hotplate. The heaters of the hotplate are switched off and the sample is left in the oil bath to cool down slowly to RT while stirring. The solids are recovered after one day by vacuum filtration to afford J-type crystals of MRE-0094.

### Example 10

### A Process for Preparing K-Type Crystal Form of MRE-0094

A saturated solution of MRE-0094 (F-type crystal form) containing excess of solids is prepared in IPA. The slurry is agitated at ˜20° C. for 30 min, following addition of heptane at 1 mL/min until a solvent ratio 1:1 v/v of heptane to IPA is reached. The slurry is agitated for approximately 30 min further, and the solids are collected by vacuum filtration, and dried at RT under vacuum to afford K-type crystals of MRE-0094.

### Example 11

### A Process for Preparing Amorphous Form of MRE-0094

A sample of MRE-0094 (A-type crystal form) is heated isothermally at approximately 180° C. until the sample melts. The sample is removed from the heat source and allowed to cool naturally at RT.

### Example 12

### Approximate Solubilities of A-Type Crystals of MRE-0094

Weighed samples of MRE-0094 (typically 30-60 mg) are treated with aliquots of the test solvent. Solvents are either reagent or HPLC grade. The aliquots are usually 200 μL or 500 μL. Between additions the mixtures are sonicated. Approximate solubilities are estimated from the total volume of solvent used to obtain a clear solution, as determined by visual inspection. Note that actual solubilities may be greater than those calculated due to the volume of the solvent aliquots or to a slow rate of dissolution. If dissolution does not occur during the experiment, the solubility is expressed as “less than”. The solubilities are reported to the nearest mg/mL.

TABLE 17

| Solvent | Solubility (mg/mL, RT) |
|---|---|
| acetone | 5 |
| ACN | <3 |
| DCM | <2 |
| 1,4-dioxane | 17 |
| DMF | 179 |
| EtOH | 7 |
| EtOH:water - 1:1 | 9 |
| EtOH:water - 19:1 | 12 |
| EtOAc | <4 |
| heptane | <4 |
| IPA | 4 |
| MeOH | 4 |
| MEK | 4 |
| THF | 23 |
| toluene | <4 |
| water | <0.3 |
| water:PG - 1:1 | 6 |
| TFE | 11 |
| PG | <71^{a} |

^{a}Calculated based on the total amount of MRE-0094 dissolved in a known amount of solvent. Known amount of MRE-0094 is agitated in the solvent for two days. The remaining solid is collected and weighed and the solubility is calculated based on the difference between starting and ending weights.

### Example 13

### Preparation of Topical Compositions

MRE-0094 is dissolved in propylene glycol in a mixing vessel. Sodium carboxymethylcellulose is slowly added to the propylene glycol mixture while stirring until lump-free. Purified water is added to a separate mixing vessel followed by the addition of the sodium acetate trihydrate, glacial acetic acid, and sodium chloride with mixing until dissolved. The purified water solution is slowly added to the propylene glycol mixture with mixing. The combined mixture is then homogenized and allowed to cool to RT. The resulting gel is filled into jars or tubes.

Quantitative formulations for MRE-0094 gels:

TABLE 18
No active agent (vehicle)

| Material | % (w/w) | Amount (mg) |
|---|---|---|
| Propylene Glycol, USP | 50 | 500 |
| Sodium Carboxymethylcellulose, USP | 1.8 | 18 |
| Sodium Acetate (trihydrate), USP | 0.15 | 1.5 |
| Glacial Acetic Acid, USP | 0.01 | 0.1 |
| Sodium Chloride, USP | 0.78 | 7.8 |
| Purified Water | 47.26 | 472.6 |
| Total | 100 | 1000 |

TABLE 19
5 μg of MRE-0094/g of gel

| Material | % (w/w) | Amount (mg) |
|---|---|---|
| MRE-0094^{a} | 0.0005 | 0.005 |
| Propylene Glycol, USP | 50 | 500 |
| Sodium Carboxymethylcellulose, USP | 1.8 | 18 |
| Sodium Acetate (trihydrate), USP | 0.15 | 1.5 |
| Glacial Acetic Acid, USP | 0.01 | 0.1 |
| Sodium Chloride, USP | 0.78 | 7.8 |
| Purified Water | 47.26 | 472.6 |
| Total | 100 | 1000 |

^{a}Corrected for purity, 2-[2-(4-chlorophenyl)ethoxy]adenosine - 97%

TABLE 20
50 μg of MRE-0094/g of gel

| Material | % (w/w) | Amount (mg) |
|---|---|---|
| MRE-0094^{a} | 0.005 | 0.05 |
| Propylene Glycol, USP | 50 | 500 |
| Sodium Carboxymethylcellulose, USP | 1.8 | 18 |
| Sodium Acetate (trihydrate), USP | 0.15 | 1.5 |
| Glacial Acetic Acid, USP | 0.01 | 0.1 |
| Sodium Chloride, USP | 0.78 | 7.8 |
| Purified Water | 47.26 | 472.6 |
| Total | 100 | 1000 |

^{a}Corrected for purity, 2-[2-(4-chlorophenyl)ethoxy]adenosine - 97%

TABLE 21
500 μg of MRE-0094/g of gel

| Material | % (w/w) | Amount (mg) |
|---|---|---|
| MRE-0094^{a} | 0.05 | 0.5 |
| Propylene Glycol, USP | 50 | 500 |
| Sodium Carboxymethylcellulose, USP | 1.8 | 18 |
| Sodium Acetate (trihydrate), USP | 0.15 | 1.5 |
| Glacial Acetic Acid, USP | 0.01 | 0.1 |
| Sodium Chloride, USP | 0.78 | 7.8 |
| Purified Water | 47.21 | 472.1 |
| Total | 100 | 1000 |

^{a}Corrected for purity, 2-[2-(4-chlorophenyl)ethoxy]adenosine - 97%

1-14. (canceled)

15. A crystal form of anhydrous MRE-0094 which crystal form (G-type crystal form) is substantially free of other polymorphic forms of MRE-0094 and has at least one of the following properties:
(a) a melting point of about 178° C. when heated at 10° C./min;
(b) a X-ray diffraction pattern with characteristic X-ray diffraction peaks at diffraction angles (2θ) of about 4.9±0.2, 6.7±0.2, 16.9±0.2, 18.5±0.2, 19.5±0.2, 20.3±0.2, 22.5±0.2 and 23.9±0.2;
(c) an infrared absorption spectrum with absorption bands at about 3470±2, 3250-3100, 3050-3020, 2980-2820, 2785-2750, 2350-2315, 1920-1875 and 1800-1780 cm⁻¹; and
(d) a Raman spectrum with Raman shifts at about 3050±2, 2970±2, 1600±2, 1510±2 (doublet), 1360-1345 (doublet) and 1310±2 cm⁻¹.

16. A crystal form according to claim 15, which crystal form has characteristic X-ray diffraction peaks at diffraction angles (2θ), and relative intensities of about:

| Angle (deg 2θ) | Relative Intensity (%) |
|---|---|
| 4.9 ± 0.2 | 83.2 |
| 6.7 ± 0.2 | 87.3 |
| 9.1 ± 0.2 | 52.3 |
| 9.9 ± 0.2 | 41.0 |
| 10.4 ± 0.2 | 46.9 |
| 11.0 ± 0.2 | 39.2 |
| 13.9 ± 0.2 | 47.2 |
| 15.6 ± 0.2 | 58.1 |
| 16.2 ± 0.2 | 48.9 |
| 16.9 ± 0.2 | 60.5 |
| 17.4 ± 0.2 | 49.0 |
| 17.8 ± 0.2 | 53.8 |
| 18.5 ± 0.2 | 72.9 |
| 19.2 ± 0.2 | 56.1 |
| 19.5 ± 0.2 | 74.5 |
| 20.3 ± 0.2 | 60.2 |
| 20.9 ± 0.2 | 57.1 |
| 21.8 ± 0.2 | 45.2 |
| 22.5 ± 0.2 | 100.0 |
| 23.5 ± 0.2 | 53.3 |
| 23.9 ± 0.2 | 63.0 |
| 24.4 ± 0.2 | 55.2 |
| 25.0 ± 0.2 | 37.9 |
| 25.6 ± 0.2 | 45.9 |
| 26.1 ± 0.2 | 41.2 |
| 26.2 ± 0.2 | 42.3 |
| 27.5 ± 0.2 | 51.5 |
| 28.3 ± 0.2 | 34.6 |
| 28.8 ± 0.2 | 36.8 |
| 29.3 ± 0.2 | 38.7 |
| 29.9 ± 0.2 | 35.4 |
| 30.2 ± 0.2 | 35.8 |
| 32.5 ± 0.2 | 31.5 |
| 33.0 ± 0.2 | 31.5 |
| 36.2 ± 0.2 | 25.4 |

17. A crystal form according to claim 15, which crystal form has all four of the properties (a), (b), (c) and (d).

18-29. (canceled)

30. A method for the production of a crystal form of 2-[2-(4-chlorophenyl)ethoxy]adenosine (MRE-0094) according to claim 15, wherein the method comprises:
(a) dissolving MRE-0094, in any of its forms, in a solvent to form a solution at a temperature ranging from room temperature (RT) to the boiling point of the solvent in which solvent MRE-0094 is suitably soluble at the dissolution temperature and in which solvent MRE-0094 is only poorly soluble at a lower temperature ranging from about −20° C. to RT;
(b) cooling the solution to the lower temperature to induce precipitation of product crystals of MRE-0094; and
(c) isolating and drying the precipitated crystals of MRE-0094.

31. (canceled)

32. The method according to claim 30, wherein the solvent is a 1:1-mixture of ethanol and water.

33. The method according to claim 30, wherein the solvent is isopropanol.

34-35. (canceled)

36. The method according to claim 30, wherein the dissolution temperature ranges from about 60° C. to about 75° C.; and the lower temperature is RT.

37-41. (canceled)

42. A method for the production of a crystal form of 2-[2-(4-chlorophenyl)ethoxy]adenosine (MRE-0094) according to claim 15, wherein the method comprises:
(a) suspending starting crystals of MRE-0094 at a temperature of at least 10° C. in a solvent in which MRE-0094 is incompletely soluble at the temperature to form the suspension;
(b) agitating the resulting suspension for a time sufficient to effect transformation of the starting crystals to product crystals of MRE-0094 at a temperature of at least 10° C. wherein the temperature to effect transformation and the temperature to form the suspension are the same or different; and
(c) isolating and drying the product crystals of MRE-0094.

43. The method according to claim 42, wherein the starting crystals of MRE-0094 are the A-type crystals.

44. The method according to claim 43, wherein the solvent is isopropanol.

45. (canceled)

46. The method according to claim 44, wherein the temperature to form the suspension and the temperature to effect transformation are both RT.

47. A method for the production of a crystal form of 2-[2-(4-chlorophenyl)ethoxy]adenosine (MRE-0094) according to claim 15, wherein the method comprises:
(a) suspending starting crystals of MRE-0094 at a temperature of at least 10° C. in a first solvent in which MRE-0094 is incompletely soluble at the temperature to form the first suspension;
(b) adding a second solvent to the first suspension at a temperature of at least 10° C. wherein the second solvent is miscible with the first solvent, and the solubility of MRE-0094 in the resulting second suspension is reduced, and wherein the temperature to form the first suspension and the temperature to form the second suspension are the same or different;
(c) agitating the resulting second suspension for a time sufficient to effect transformation of the starting crystals to product crystals of MRE-0094 at a temperature of at least 10° C. wherein the temperature to effect transformation and the temperature to form the second suspension are the same or different; and
(d) isolating and drying the product crystals of MRE-0094.

48. The method according to claim 47, wherein the first solvent is isopropanol and the second solvent is heptane.

49. The method according to claim 48, wherein the starting crystals of MRE-0094 are the A-type crystals.

50. A method for the production of the G-type crystal form of 2-[2-(4-chlorophenyl)ethoxy]adenosine (MRE-0094) substantially free of other polymorphic forms of MRE-0094, wherein the method comprises:
(a) dissolving MRE-0094, in any of its forms, in a first solvent to form a solution at a dissolution temperature of about the boiling point of the solvent in which solvent MRE-0094 is readily soluble at the dissolution temperature;
(b) if desired adding G-type seed crystals to the solution of MRE-0094 in the first solvent;
(c) cooling the mixture gradually to a lower temperature of about 10° C. to induce precipitation;
(d) treating the mixture with a second solvent which is miscible with the first solvent, and in which MRE-0094 is only poorly soluble to further induce precipitation of G-type crystals of MRE-0094, and wherein the ratio of the first solvent to the second solvent in the final mixture ranges from about 1:1 to about 1:2 by volume;
(e) agitating the resulting suspension for a time sufficient to complete precipitation of the G-type crystals of MRE-0094; and
(f) isolating and drying the precipitated G-type crystals of MRE-0094.

51. The method according to claim 50, wherein the first solvent is isopropanol, the second solvent is heptane, and the dissolution temperature is about 85° C.

52. The method according to claim 51, wherein the ratio of the first solvent to the second solvent is about 1:2 by volume.

53. The method according to claim 52, wherein cooling the mixture gradually in step (c) comprises:
(1) cooling the mixture at rate of about 0.1° C./min from about 85° C. to about 50° C.;
(2) agitating the mixture at 50° C. for about 4 h;
(3) cooling the mixture at rate of about 0.1° C./min from about 50° C. to about 10° C.; and
(4) agitating the mixture at 10° C. for about 3 h.
